(19) United States
(12) Patent Application Publication
(10) Pub. No.: US 2010/0047775 A1
(43) Pub. Date: Feb. 25, 2010

(54) Method of identifying individuals at risk of perioperative myocardial injury, major adverse cardiac events, cognitive decline, arrhythmias, depression or bleeding

(76) Inventors: Debra A Schwinn, Seattle, WA (US); Mihai V. Podgoreanu, Durham, NC (US); Joseph P. Mathew, Durham, NC (US); Hilary P. Grocott, Durham, NC (US); William D. White, Durham, NC (US); Richard W. Morris, Durham, NC (US); Mark F. Newman, Durham, NC (US); Barbara Phillips-Bute, Durham, NC (US); Ian Welsby, Durham, NC (US)

Correspondence Address: NIXON & VANDERHYE, PC, 901 NORTH GLEBE ROAD, 11TH FLOOR, ARLINGTON, VA, 22203, US

(21) Appl. No.: 11/919,008

(22) PCT Filed: Apr. 24, 2006
(86) PCT No.: PCT/US2006/015266

§ 371 Date: Oct. 26, 2009

Related U.S. Application Data
(60) Provisional application No. 60/673,778, filed on Apr. 22, 2005.
(60) Provisional application No. 60/680,037, filed on May 12, 2005.
(60) Provisional application No. 60/693,052, filed on Jun. 23, 2005.
(60) Provisional application No. 60/775,783, filed on Feb. 23, 2006.
(60) Provisional application No. 60/781,755, filed on Mar. 14, 2006.
(60) Provisional application No. 60/781,754, filed on Mar. 14, 2006.

(51) Int. Cl. C12Q 1/68; C07H 21/04

(52) U.S. Cl. 435/6; 536/24.3; 536/24.33

(57) ABSTRACT

The present invention relates to methods of identifying individuals at risk of perioperative myocardial injury, major adverse cardiac events, cognitive decline, arrhythmias, depression and bleeding.

This application claims priority from U.S. Provisional Application 60/673,778, filed Apr. 22, 2005, U.S. Provisional Application 60/680,037, filed May 12, 2005, U.S. Provisional Application 60/693,052, filed Jun. 23, 2005, U.S. Provisional Application 60/781,755, filed Mar. 14, 2006, U.S. Provisional Application 60/775,783, Feb. 23, 2006, and U.S. Provisional Application 60/781,754, filed Mar. 14, 2006, the entire disclosures of which are incorporated herein by reference.

This invention was made with Government support under RO1 AG17556 awarded by the National Institutes of Health. The Government has certain rights in the invention.

### TECHNICAL FIELD

The present invention relates to methods of identifying individuals at risk of perioperative myocardial injury, major adverse cardiac events, cognitive decline, arrhythmias, depression or bleeding.

### BACKGROUND

Surgery, like accidental trauma, triggers a complex inflammatory host response. This is greatly magnified in cardiac surgery because of continuous exposure of blood to nonendothelial surfaces characteristic of cardiopulmonary bypass (CPB). The acute inflammatory response to CPB is a primary mechanism in the pathogenesis of perioperative myocardial injury, which is a multifactorial disorder with significant interpatient variability. Despite the substantial advances in surgical, cardioprotective and anesthetic techniques, the incidence of perioperative myocardial infarction following cardiac surgery has remained at 7-15% (Mangano et al, JAMA 277:325-32 (1997)), and is associated with reduced long-term survival (Force et al, Circulation 82:903-12 (1990)).

The mechanism underlying the variability in myocardial injury remains poorly predicted by clinical, procedural, and biological markers. However, increased evidence for heritability of the pro-inflammatory state (Pankow et al, Atherosclerosis 154:681-9(2001)), coupled with numerous reports from genetic association studies, suggest that the individual genetic background modulates the magnitude of postoperative systemic inflammatory response following cardiac surgery.

The present invention results, at least in part, from studies designed to test the hypothesis that single nucleotide polymorphisms (SNPs) in selected inflammatory genes are associated with the incidence of myocardial injury following cardiac surgery. These studies have resulted in the identification of specific polymorphisms that modulate the risk of myocardial injury following surgery.

Significant differences exist between males and females in the susceptibility to coronary artery disease, its time progression and treatment outcomes. Women are reported to be at increased risk for poor outcome after coronary artery bypass grafting (CABG). Particularly, major adverse cardiac events (MACE), including myocardial infarction, target vessel revascularization, cardiac arrest, cardiac and all-cause mortality, occur more often in the female population undergoing CABG surgery (Edwards et al, Ann. Thor. Surg. 66(1):125-131 (1998), Vaccarino et al, Circulation 105(10):1176-1181 (2002)). In part these differences are explained by dissimilar preoperative profiles, including smaller vessels and body size, and higher prevalence of hypertension, insulin dependent diabetes mellitus, and congestive heart failure (Koch et al, J. Thor. Cardiovas. Surg. 126(6):2044-2051 (2003), Guru et al, J. Thor. Cardiovas. Surg. 127(4):1158-1165 (2004)). However, despite the adjustment for these factors, multivariate analysis studies still demonstrate worse survival in women up to 2-3 years post CABG surgery (Guru et al, J. Thor. Cardiovas. Surg. 127(4):1158-1165 (2004), King et al, JAMA 291(10):1220-1225 (2004), Risum et al, Eur. J. Cardio-Thor. Surg. 11(3):539-546 (1997)).

The present invention further results, at least in part, from studies designed to study genetic makeup of cardiac surgery patients as the tool to reveal gender related genotypes associated with poor outcome. This approach has been recently successfully implemented to assess the gender-related risk of major cardiovascular complications in nonsurgical patients with coronary artery disease (Yamada et al, New Engl. J. Med. 347(24):1916-1923 (2002)). In order to characterize the surgical patients, genetic markers known to have a functional role in complex processes of surgical stress and cardiovascular response were analyzed. Of particular interest were interactions created by inflammatory response, endothelial and platelet activity, and autonomic and paracrine control. With the use of public databases (McCarthy et al, J. Med. Genet. 41(5):334-341 (2004)), including PubMed and Online Mendelian Inheritance in Man, candidate genes were selected that have been characterized and potentially associated with MACE outcomes from cardiac surgery and/or from a progression of coronary artery disease. As a next step, polymorphisms of these genes were selected that have been characterized to change the phenotypic expression, i.e., function or level of expression, of the encoded protein. The focus was on 1-3 common single nucleotide polymorphisms or insertion/deletions in the coding region per gene. Also studied were data reported on gender based differences for candidate genes and their polymorphisms. As a result of this examination, a set group of polymorphisms was preselected for subsequent analysis.

Postoperative cognitive dysfunction, encompassing impairments in attention, memory, language, and processing time, serves as a marker of long-term cognitive decline and is associated with a reduced quality of life despite patient's expectations that recovery of physical status will generally improve their lives (Newman et al, Stroke 32:2874-2881 (2001)). Although many adverse outcomes related to cardiac surgery have been minimized, little progress has been made in reducing cognitive dysfunction, occurring in as many as 53% of patients at hospital discharge, 36% at six weeks, 24% at six months and 42% at five years (Newman et al, N. Engl. J. Med. 344:395-402 (2001)). Of all the clinical predictors of cognitive decline after surgery, only advanced age, baseline cognition, and level of education consistently portend postoperative deficits.

The inability to derive a widely applicable predictive model, coupled with preliminary reports of heritability of cognitive decline (Lee, J. Clin. Exp. Neuropsychol. 25:594-613 (2003), Potter et al, Neurology 63:2245-2249 (2004)), suggest that a genetic contribution modulates response to this type of neurological injury. Preliminary evidence of an association between the presence of the apolipoprotein E-εA allele and risk of cognitive impairment at 6 weeks after surgery was reported previously (Tardiff et al, Ann. Thoract. Surg. 64:715-720 (1997)). Similarly, in a pilot study, the PL^{A2} polymorphism of the GPIIIa constituent of the platelet integrin receptor, GPIIb/IIIa, was associated with more severe early postoperative cognitive decline when compared with PL^{A1} homozygotes (Mathew et al, Ann. Thorac. Surg. 71:663-666 (2001)). However, both studies were limited by small sample size and the examination of only a single polymorphism.

The etiology of perioperative neurological injury is multifactorial and includes cerebral embolism and hypoperfusion but it is also clear that inflammatory processes related to cardiopulmonary bypass or the surgical procedure itself contribute to the insult (Mathew et al, Stroke 34:508-513 (2003)). Because of the complex nature of cognitive decline, a number of genotypes may be involved as disease modifiers.

The present invention additionally results, at least in part, from studies designed to test the hypothesis that gene polymorphisms in biological pathways regulating inflammation, cell matrix adhesion/interaction, coagulation-thrombosis, lipid metabolism and vascular reactivity are associated with the incidence of cognitive dysfunction following cardiac surgery. The invention provides methods of identifying individuals at risk of perioperative cognitive decline.

Perioperative arrhythmias are a common problem for cardiac surgery patients and are associated with substantial increases in postoperative complication, prolonged hospital stay, and reductions in long-term survival. Despite many advances in the field of cardiac surgery, patients remain at risk for perioperative arrhythmias of many kinds.

Prolongation of corrected QT (QTc—a specific electrical abnormality) interval has been associated with risk of cardiovascular adverse events in a broad range of clinical populations (Okin, J. Am. Coll. Cardiol. 43(4):572-574 (2004)), including patients undergoing non-cardiac surgery (Anderson, Cardiothorac. Vasc. Anesth. 18(3):281-287 (2004)). Although long-term changes in ventricular repolarization following coronary revascularization have been reported (Gulcan, Am. Heart J. 149:917-920 (2005), Wozniak-Skowerska, Med. Sci. Monit. 10(3):CR128-131 (2004)), the impact of cardiac surgical injury on early postoperative repolarization abnormalities is not known.

The present invention yet further results, at least in part, from studies designed to test the hypothesis that clinical, procedural and genetic factors are associated with perioperative changes in QTc interval in cardiac surgical patients. These studies have resulted in the identification of a genetic component to the incidence and severity of prolongation of QTc after heart surgery. The invention provides a method for effecting the preoperative identification of high-risk cardiac surgical patients and makes possible the development of novel cardioprotective strategies.

Depression is prevalent, enormously expensive (Greenberg et al, J. Clin. Psych. 64:1465-75 (2003)), and has multiple complex causes (Wong et al, Nat. Rev. Drug Disc. 3:136-51 (2004)). According to the NIMH, of the nearly 35 million Americans age 65 and older, an estimated 2 million are depressed (Health NIoM: Older Adults: Depression and Suicide Facts. Bethesda, Md., National Institutes of Health, US Department of Health and Human Services (2003)). Most of them do not seek help, and, consequently, depression is both under-diagnosed and under-treated. Contributing causal factors include traumatic or stressful life events, serious medical conditions, and family history.

Research over the past two decades shows strong associations between depression and cardiac health. Depression has been shown to be an independent risk factor for coronary artery disease (CAD) (Wulsin et al, Psych. Med. 65:201-10 (2003)), and can be a precursor to heart attacks (Pratt et al, Circulation 94:3123-9 (1996)) or cardiac death (Penninx et al, Arch. Gen. Psych. 58:221-7 (2001)). Patients who are depressed after acute myocardial infarction (MI) are at increased risk for mortality or additional cardiac events (Frasure-Smith et al, Circulation 101:1919-24 (2000)). In addition, depression is significantly associated with increased cardiac hospitalization, and poor quality of life in the first year after MI (Bush et al, Post-Myocardial Infarction Depression. Summary, Evidence Report/Technology Assessment: Number 123. AHRQ Publication Number 05-EO18-1 Agency for Healthcare Research and Quality, Rockville, Md. (2005)).

Coronary artery bypass graft (CABG) is a common surgical intervention for CAD patients, and depression rates are high before and after surgery (Connerney et al, Lancet 358:1766-71 (2001); Lett et al, Psych. Med. 66:305-15 (2004)). Depressed patients are more than twice as likely to have a cardiac event within 12 months after surgery. Depression on the day before surgery as well as depression that persists until 6 months after surgery is associated with 2-3 fold increased risk of mortality. Even patients who are mildly depressed 6 months after coronary artery bypass surgery are at increased risk for mortality (Blumenthal et al, Lancet 362:604-9 (2003)).

Serotonin (a monoamine neurotransmitter) has been implicated in multiple mood disorders, including depression. The monoamine hypothesis of depression began almost 50 years ago when the first antidepressants were shown to increase concentrations of monoamine neurotransmitters in the brain, resulting in elevated moods among psychiatric patients. The monoamine hypothesis speculates that mood disorders are caused by a deficiency of monoamines at receptor sites in the brain, creating a chemical imbalance (Castren, Nat. Rev. Neur. 6:241-6 (2005)). Genetic studies of depression, as applied to serotonergic function, extend this theory by searching for the genetic polymorphisms which encode the proteins that mediate the synthesis of serotonin, its reuptake after release, its effect on pre-and post synaptic neurons, and its breakdown (Jonsson et al, BMC Psychiatry 4:4 (2004), Wong et al, Nat. Rev. Drug Disc. 3:136-51 (2004)).

MAOA-uVNTR is a polymorphism in the upstream regulatory region of the gene encoding monoamine oxidase A (MAOA), which deaminates serotonin and norepinephrine (Williams et al, Neuropsychopharmacology 28:533-41 (2003)). MAOA has been found to be associated with higher levels of aggressiveness/impulsivity (Manuck et al, Psych. Res. 95:9-23 (2000)), with bi-polar disorder among females (Craddock et al, Bipolar Dis. 3:284-98 (2001); Preisig et al, Am. J. PharmacoGenomics 5:45-52 (2005)), aggressive driving (Li et al, Ch. J. Prev. Med. 38(5):321-3 (2004)) and major depression and sleep disturbance among males (Du et al, Neuroreport 15:2097-101 (2004)).

5HTTLPR is a 44-base pair insertion/deletion polymorphism in the 5′ flanking regulatory region of the serotonin transporter gene that is associated with differential transcriptional efficiencies, characterized by either long or short alleles (Lesch et al, Science 274:1527-31 (1996)). The short (S) allele has been shown to be associated with increased levels of neuroticism, decreased levels of agreeableness, and negative moods in men (Williams et al, Neuropsychopharmacy 28:533-41 (2003)). The L/L (both DNA strands having the long allele) genotype has been shown to be associated with lower levels of anxiety, depression, impulsivity, and hostility (Lesch et al, Science 274:1527-31 (1996)). On the other hand, the S allele has been associated with early-onset alcoholism and impulsive violent behavior in a European sample, and alcohol-related anti-social behavior in a Japanese sample. The L allele is associated with greater cardiovascular responses to stress (Williams et al, Psych. Med. 63:300-5 (2003)).

Because of the strong association between depression in cardiac patients and poor outcome, an assessment was made as to whether genetic variability in two serotonin-related gene polymorphisms predicts new onset depression in CABG patients. The present invention additionally provides genetic markers that can be used to identify patients at risk for depression and thereby anticipate patients who may be at risk for poor outcome but for whom other pre-operative predictors are not available. Of particular interest is new onset depression, or depression that cannot be predicted from baseline depression scores.

Microvascular bleeding remains a major problem following cardiac surgery with cardiopulmonary bypass (CPB) (Nuttall et al, Anesthesiology 94:773-781, discussion 5A-6A (2001), Hall et al, Cardiovasc. Surg. 10:146-153 (2002)),with up to 5% of patients receiving more than a 10 unit perioperative blood transfusion (Woodman and Harker, Blood 76:1680-1697 (1990)). Approximately 4% of patients require reoperation for hemorrhage (Hall et al, Cardiovasc. Surg. 10:146-153 (2002), Woodman and Harker, Blood 76:1680-1697 (1990)) which is associated with increased mortality and morbidity (Unsworth-White et al, Anannls of Thoracic Surgery 59:664-667 (1995)). Current risk stratification based on clinical, procedural, and biological markers (Wahba et al, Journal of Cardiothoracic & Vascular Anesthesia 11:824-827 (1997), Despotis et al, Anesthesia & Analgesia 82:13-21 (1996)) has been only partially successful, failing to account for much of the postoperative blood loss seen even with “low-risk” primary coronary artery bypass (CABG) surgery (Hardy et al, Canadian Journal of Anaesthesia 38:511-517 (1991)). CPB-induced alterations in the hemostatic system are multifactorial, pertaining to excessive activation of coagulation and fibrinolytic pathways with interplay of cellular and soluble hemostatic and inflammatory systems; hypothermia and hemodilution further complicate the situation (Despotis et al, Annals of Thoracic Surgery 72:S1821-1831 (2001)). Coagulopathy following CPB represents one extreme on a continuum of coagulation function, with perioperative prothrombotic outcomes (e.g. coronary graft thrombosis, myocardial infarction, stroke and pulmonary embolism) at the other end of the spectrum (Spiess and Chandler, Best Practice & Research: Clinical Anaesthesiology 15:195-211 (2001)).

Pathophysiologically, the balance between bleeding, normal hemostasis, and thrombosis is markedly influenced by the rate of thrombin formation and platelet activation (Kunicki and Nugent, Vox Sang. 83 (Suppl 1):85-90 (2002), Slaughter et al, Anesthesiology 80:520-526 (1994)). There is recent evidence that genetic variability modulates the activity in each of these mechanistic pathways (Spiess and Chandler, Best Practice & Research: Clinical Anaesthesiology 15:195-211 (2001)). However, little is known of the role of allotypic coagulation, fibrinolytic and platelet-membrane receptor gene variation in predicting bleeding following CABG surgery; the few studies to date focus only on single-gene variants (Donahue et al, Circulation 107(7):1003-1008 (2003)). Several prothrombotic genetic polymorphisms are known to exist.

The present invention yet additionally results, at least in part, from studies designed to test the hypothesis that genetic polymorphisms of cytokines and cellular adhesion molecules are associated with bleeding after cardiac surgery. The invention provides a method of identifying patients with a E-selectin/ELAM-1 polymorphism, which polymorphism is associated with increased postoperative bleeding.

### SUMMARY OF THE INVENTION

The present invention relates generally to perioperative myocardial injury, major adverse cardiac events, cognitive decline, arrhythmias, depression and bleeding. More specifically, the invention relates to methods of identifying individuals at risk of perioperative myocardial injury, major adverse cardiac events (MACE), cognitive decline, arrhythmias, depression or bleeding, and to compositions and kits suitable for use in such methods.

Objects and advantages of the present invention will be clear from the description that follows.

### BRIEF DESCRIPTION OF THE DRAWINGS

FIG. 1. Chromosomal distribution of candidate genes for myocardial injury.

FIG. 2. Polymorphisms associated with increased risk of myocardial injury.

FIG. 3. Polymorphisms protective against myocardial injury.

FIG. 4. Kaplan-Meier survival curves for patients undergoing combined CABG-valve procedures by gender and SELP 1902A/G genotypes.

FIG. 5. Incidence of postoperative cognitive deficit by CRP 1059G/C and SELP 1087G/A genotypes. The incidence of cognitive deficit was 16.7% in carriers of minor alleles at both of these loci compared to 43.0% in patients homozygous for the major allele.

FIG. 6. Median CRP levels by CRP 1059G/C genotypes. CRP levels at the 24-hour sampling time in patients homozygous (CC) or heterozygous (CG) for the minor allele were lower compared to patients homozygous (GG) for the major allele. * P<0.05

FIG. 7. Median platelet activation (indexed to baseline) by SELP 1087G/A genotypes. Platelet activation was significantly lower upon release of the cross clamp in patients with the homozygous minor (AA) or heterozygous (GA) genotype compared to patients homozygous (GG) for the major allele. * P<0.05

FIG. 8. MAO-A (MAOA) genotypes and depression (CESD>=16) at preoperative baseline, 6 months and 1 year after surgery.

FIG. 9. Serotonin transport genotypes (categorized as the absence or presence of the short allele) and depression.

FIG. 10. Carriers of one (GT) and two (TT) minor alleles show a gene dose effect in terms of an increase in bleeding (p=0.01) expressed as a percentage increase in chest tube drainage over the first 4 postoperative hours compared to the wild type genotype (GG).

FIG. 11. More blood products were transfused perioperatively in carriers of the minor allele (98GT and 98TT) compared to patients of wild type genotype (98GG). The amount of fresh frozen plasma (FFP) transfused was significantly different (p=0.03). The data are expressed as means because the median values were zero.

### DETAILED DESCRIPTION OF THE INVENTION

In one embodiment, the present invention results from studies designed to examine the association between specific genetic polymorphisms and myocardial injury risk after surgery (e.g., cardiac surgery). These studies demonstrate that specific genetic variants contribute to the risk of postoperative myocardial injury and suggest that inflammation plays a pivotal role.

It will be appreciated from a reading of this disclosure that the presence of certain single nucleotide polymorphisms (SNPs, genetic variants) in the genes for C-Reactive Protein (CRP), Lipopolysaccharide Binding Protein (OBP), Interleukin 6 (IL6), E-Selectin (Endothelial Leukocyte Adhesion Molecule 1) (ELAM1), Catalase (CAT) and Intercellular Adhesion Molecule 1 (ICAM1) are predictive of perioperative myocardial injury. Presence of the minor allele at four of these loci had a deleterious effect (CRP 1846T OR 2.4 [95% CI 1.1-5.1]; LBP 19983C OR 2.5 [1.2-4.8]; IL6-572C OR 2.4 [1-5.9], ICAM1 1462G), and was protective at the other two loci (ELAM1 98T OR 0.1[0.02-0.9]; CAT-844T OR 0.5[0.3-0.8]).

Biological effects for the single nucleotide polymorphisms (SNPs) referenced above, and described in greater detail in Examples 1 and 2 that follow, have been demonstrated. This embodiment of the present invention provides definitive association between these genetic variants and clinical postoperative myocardial injury in the perioperative setting. This embodiment is exemplified by reference to cardiac surgery patients but includes all perioperative, periprocedure (endoscopy, bronchoscopy, cardiac catheterization, angioplasty, etc.), and intensive care unit settings.

The presence of one or more of the above-referenced polymorphisms present in a sample (e.g., a biological sample such as blood) can be determined using any of a variety of genotyping techniques known in the art. Examples of such techniques include the use of polymerase chain reaction and extension primers (see too the Example below). Suitable techniques also include the use of RFLP analysis and mass spectrometry (see also Ye et al, Hum. Mutat. 17(4):305 (2001), Chen et al, Genome Res. 10:549 (2000)).

The genetic variants (SNPs) described above and in Examples 1 and 2 can be used, for example, to predict postoperative and ICU myocardial injury risk. As indicated above, screening for genetic variants of the invention is also relevant for other invasive procedures including but not limited to endoscopy, bronchoscopy, cardiac catheterization, and angioplasty. Preoperative screening for genetic variants enables clinicians to better stratify a given patient for therapeutic intervention, either with drug therapy or with other modalities. Additionally, knowledge of genetic variants allows patients to choose, in a more informed way in consultation with their physician, medical versus procedural therapy. Identifying these genetic variants in patients who decide to undergo surgery or other invasive procedure enables health care providers to design altered therapeutic strategies aimed at preventing the incidence of myocardial injury in the subset of patients with enhanced risk. In addition, identifying these genetic variants in patients who have already experienced myocardial injury might also lead to alteration or modification in the therapeutic strategy.

As indicated above, preoperative genotype testing can refine risk stratification and improve patient outcome. Based on the genetic risk factors identified, drugs already available and used to minimize the risk of myocardial injury and/or damage associated therewith (e.g., beta blocker drugs such as metoprolol (to decrease energy expenditure of myocardium and provide protection), opioid drugs (to stimulate delta opioid receptors that are known to mediate myocardial protection), chemical preconditioning drugs such as ATP-sensitive K+ channel activators, the NHE1 inhibitor, cariporide, etc.) can be useful in reducing myocardial injury risk/damage in acute settings, for example, cardiac surgery. Identification of the genetic markers described herein can facilitate individually tailored medical therapy (personalized medicine) designed to reduce myocardial injury risk and associated morbidity and mortality. Perioperative screening can facilitate alterations in the usual course of the surgical procedure with institution of procedures designed to additionally reduce this risk (examples of changes that could be made in surgical procedure include: perform off-pump surgery rather than having heart surgery with cardiopulmonary bypass, provide ischemic preconditioning—a brief period of ischemia to precondition the heart against further ischemic injury (usually 5 minutes with a rest period before aortic cross clamping), minimize aortic cross-clamp/myocardial ischemic time, possibly not cross-clamp at all, provide blood cardioplegia, etc.).

In a further aspect of this embodiment, the present invention relates to methods of identifying compounds suitable for use in minimizing the risk of myocardial injury. These methods can comprise screening compounds for their ability to modulate (e.g., inhibit) inflammation (e.g., perioperative inflammation). Such methods are made possible by the identification of functional genetic variants, occurring in genes involved in leukocyte-endothelial interaction, endotoxin responsiveness, and free radical injury pathways, which are independent risk factors modulating the severity of myocardial injury after cardiac surgery.

In another embodiment, the present invention results from studies designed to examine the association between specific genetic polymorphisms and MACE after surgery (e.g., cardiac surgery). These studies demonstrate that specific genetic variants contribute to the risk of postoperative MACE, particularly in women.

It will be appreciated from a reading of this disclosure that a highly significant interaction between gender and the SELP 1902A/G (rs6127) polymorphism was found (p<0.001) in patients undergoing CABG surgery with any type of valve surgery (CV group). Female carriers of the minor allele experienced significantly more MACE when compared to other groups (hazard ratio of 18.5; 95% CI (confidence interval) 3.4-99.7). This significant finding persisted in multivariable regression modeling adjusting for age or Charlson comorbidity score (p<0.001). That is, the risk of MACE after combined CABG and valvular surgery in women was significantly associated with a SNP in the P-selectin gene.

Biological effects for the single nucleotide polymorphism (SNP) referenced above, and described in greater detail in Example 3 that follows, have been demonstrated. This embodiment of the present invention provides definitive association between this genetic variant and clinical postoperative MACE in women in the perioperative setting. This embodiment is exemplified by reference to cardiac surgery patients but includes all perioperative, periprocedure (endoscopy, bronchoscopy, cardiac catheterization, angioplasty, etc.), and intensive care unit settings.

The presence of the above-referenced polymorphism present in a sample (e.g., a biological sample such as blood) can be determined using any of a variety of genotyping techniques known in the art. Examples of such techniques include the use of polymerase chain reaction and extension primers (see too the Example below). Suitable techniques also include the use of RFLP analysis and mass spectrometry (see also Ye et al, Hum. Mutat. 17(4):305 (2001), Chen et al, Genome Res. 10:549 (2000)).

The genetic variant (SNP) described above and in Example 3 can be used, for example, to predict postoperative and ICU MACE risk, particularly in women. As indicated above, screening for the genetic variant of the invention is also relevant for other invasive procedures including but not limited to endoscopy, bronchoscopy, cardiac catheterization, and angioplasty. Preoperative screening for the genetic variant enables clinicians to better stratify a given patient for therapeutic intervention, either with drug therapy or with other modalities. Additionally, knowledge of the genetic variant allows patients to choose, in a more informed way in consultation with their physician, medical versus procedural therapy. Identifying this genetic variant in patients who decide to undergo surgery or other invasive procedure enables health care providers to design altered therapeutic strategies aimed at preventing the incidence of MACE in the subset of patients with enhanced risk. In addition, identifying this genetic variants in patients who have evidence of sensitivity toward myocardial injury (e.g., angina or a family history of myocardial injury/events) might also lead to alteration or modification in the therapeutic strategy.

As indicated above, preoperative genotype testing can refine risk stratification and improve patient outcome. Based on the genetic risk factor identified, drugs already available and used to minimize the risk of MACE and/or damage associated therewith (e.g., beta blocker drugs such as metoprolol (to decrease energy expenditure of myocardium and provide protection), opioid drugs (to stimulate delta opioid receptors that are known to mediate myocardial protection), chemical preconditioning drugs such as ATP-sensitive K+ channel activators, the NHE1 inhibitor, cariporide, etc.) can be useful in reducing myocardial injury risk/damage in acute settings, for example, cardiac surgery. Identification of the genetic marker described herein can facilitate individually tailored medical therapy (personalized medicine) designed to reduce MACE risk and associated morbidity and mortality. Perioperative screening can facilitate alterations in the usual course of the surgical procedure with institution of procedures designed to additionally reduce this risk (examples of changes that could be made in surgical procedure include: perform off-pump surgery rather than having heart surgery with cardiopulmonary bypass, provide ischemic preconditioning—a brief period of ischemia to precondition the heart against further ischemic injury (usually 5 minutes with a rest period before aortic cross clamping), minimize aortic cross-clamp/myocardial ischemic time, possibly not cross-clamp at all, provide blood cardioplegia, etc.).

In a further aspect of this embodiment, the present invention relates to methods of identifying compounds suitable for use in minimizing the risk of MACE. These methods can comprise screening compounds for their ability to modulate (e.g., inhibit) inflammation (e.g., perioperative inflammation). Such methods are made possible by the identification of a functional genetic variant, occurring in a gene involved in inflammation, which is an independent risk factor modulating the severity of MACE after cardiac surgery.

In another embodiment, the present invention results from studies designed to examine the association between specific genetic polymorphisms and cognitive decline following surgery (e.g., cardiac surgery). These studies demonstrate that specific genetic variants contribute to a reduced risk of postoperative cognitive decline.

Polymorphisms found to be significantly associated with a reduction in cognitive deficit are a CRP (1059 G/C) SNP and a SELP (1087 G/A) SNP. These polymorphisms were also found to be associated with reductions in serum CRP and platelet activation.

Biological effects for the single nucleotide polymorphisms (SNPs) referenced above, and described in greater detail in Example 4 that follows, have been demonstrated. This embodiment of the present invention provides definitive association between these genetic variants and a reduction in clinical postoperative cognitive dysfunction in the perioperative setting. This embodiment is exemplified by reference to cardiac surgery patients but includes all perioperative, periprocedure (endoscopy, bronchoscopy, cardiac catheterization, angioplasty, etc.), and intensive care unit settings.

The presence of the above-referenced polymorphisms present in a sample (e.g., a biological sample such as blood) can be determined using any of a variety of genotyping techniques known in the art. Examples of such techniques include the use of polymerase chain reaction and extension primers (see too the Example below). Suitable techniques also include the use of RFLP analysis and mass spectrometry (see also Ye et al, Hum. Mutat. 17(4):305 (2001), Chen et al, Genome Res. 10:549 (2000)).

The genetic variants (SNPs) described above and in Example 4 can be used, for example, to predict postoperative and ICU cognitive decline risk. As indicated above, screening for the genetic variants of the invention is also relevant for other invasive procedures including but not limited to endoscopy, bronchoscopy, cardiac catheterization, and angioplasty. Preoperative screening for the genetic variants enables clinicians to better stratify a given patient for therapeutic intervention, either with drug therapy or with other modalities. Additionally, knowledge of the genetic variants allows patients to choose, in a more informed way in consultation with their physician, medical versus procedural therapy. Screening for these genetic variants in patients who decide to undergo surgery or other invasive procedure enables health care providers to identify individuals who might benefit from altered therapeutic strategies aimed at preventing the incidence of cognitive dysfunction. In addition, genetic screening of patients who have evidence of sensitivity toward neurocognitive decline, or a family history, might also lead to alteration or modification in the therapeutic strategy.

As indicated above, preoperative genotype testing can refine risk stratification and improve patient outcome. Based on the genetic risk factor identified, drugs used to minimize the risk of cognitive dysfunction (e.g., pexelizumab and donepezil) can be useful in reducing cognitive dysfunction risk in acute settings, for example, cardiac surgery. Screening for the genetic markers described herein can facilitate individually tailored medical therapy (personalized medicine) designed to reduce cognitive decline risk. Perioperative screening can facilitate alterations in the usual course of the surgical procedure with institution of procedures designed to additionally reduce this risk (examples of changes that could be made in surgical procedure include: perform off-pump surgery rather than having heart surgery with cardiopulmonary bypass, and avoid aortic cross-clamping and aggressively prevent hyperthermia and hyperglycemia).

In a further aspect of this embodiment, the present invention relates to methods of identifying compounds suitable for use in minimizing the risk of cognitive dysfunction. These methods can comprise screening compounds for their ability to modulate (e.g., inhibit) thrombosis and cell matrix adhesion. Such methods are made possible by the identification of genetic variants, occurring in genes involved in inflammation and cell matrix adhesion/interaction.

In a still further embodiment, the present invention results from studies designed to examine the association between specific genetic polymorphisms and arrhythmia risk after surgery (e.g., cardiac surgery). These studies demonstrate that specific genetic variants contribute to the risk of postoperative arrhythmias and suggest that inflammation and adrenergic responsiveness play a pivotal role. It will be appreciated from a reading of this disclosure that SNPs in β-2 adrenergic receptor (ADRB2) and interleukin-1β (IL1B) genes independently associate with postoperative QTc prolongation.

Biological effects for the single nucleotide polymorphisms (SNPs) referenced above, and described in greater detail in Example 5 that follows, have been demonstrated. This embodiment of the present invention provides definitive association between these genetic variants and clinical postoperative prolonged QTc and arrhythmias in the perioperative setting. This embodiment is exemplified by reference to cardiac surgery patients but includes all perioperative, periprocedure (endoscopy, bronchoscopy, cardiac catheterization, angioplasty, etc.), and intensive care unit settings. The presence of one or more of the above-referenced polymorphisms present in a sample (e.g., a biological sample such as blood) can be determined using any of a variety of genotyping techniques known in the art (e.g., using a preoperative “CHIP” or SNP panel). Examples of such techniques include the use of polymerase chain reaction and extension primers (see too the Example below). Suitable techniques also include the use of RFLP analysis and mass spectrometry (see also Ye et al, Hum. Mutat. 17(4):305 (2001), Chen et al, Genome Res. 10:549 (2000)). (Preferred primers (forward, reverse and extension) for ADRB2 rs1800888 are:
ACGTTGGATGAGTGCATCTGAATGGGCAAG
ACGTTGGATGTGCTGACCAAGAATAAGGCC
CATCTGAATGGGCAAGAAGGAG, and for IL1B rs16944 are:
ACGTTGGATGATTTTCTCCTCAGAGGCTCC
ACGTTGGATGTGTCTGTATTGAGGGTGTGG
TGCAATTGACAGAGAGCTCC.

The genetic variants (SNPs) described above and in Example 5 can be used, for example, to predict postoperative and ICU arrhythmia risk. As indicated above, screening for genetic variants of the invention is also relevant for other invasive procedures including but not limited to endoscopy, bronchoscopy, cardiac catheterization, and angioplasty. Preoperative screening for genetic variants enables clinicians to better stratify a given patient for therapeutic intervention, either with drug therapy or with other modalities. Additionally, knowledge of genetic variants allows patients to choose, in a more informed way in consultation with their physician, medical versus procedural therapy. Identifying these genetic variants in patients who decide to undergo surgery or other invasive procedure enables health care providers to design altered therapeutic strategies aimed at preventing the incidence of arrhythmias in the subset of patients with enhanced risk. In addition, identifying these genetic variants in patients with prolonged QT at any stage of life may also lead to alteration or modification in the therapeutic (e.g., drug) strategy/therapy.

As indicated above, preoperative genotype testing can refine risk stratification and improve patient outcome. Based on the genetic risk factors identified, drugs already available and used to minimize the risk of arrhythmias and/or damage associated therewith (e.g., modulators of β-adrenergic responsiveness and anti-inflammatory agents) can be useful in reducing arrhythmia risk in acute settings, for example, cardiac surgery. Identification of the genetic markers described herein can facilitate individually tailored medical therapy (personalized medicine) designed to reduce arrhythmia risk and associated morbidity and mortality. Perioperative screening can facilitate alterations in the usual course of the surgical procedure with institution of procedures designed to additionally reduce this risk (e.g., altering cardioplegia techniques to minimize the extent of electrophysiological abnormalities associated with responses to cardiac surgical injury, etc.; alternatively, using an antiarrhythmic agent preemptively, depending on the patient's situation).

In a further aspect of this embodiment, the present invention relates to methods of identifying compounds suitable for use in minimizing the risk of arrhythmia. These methods can comprise screening compounds for their ability to modulate (e.g., inhibit) inflammation (e.g., perioperative inflammation) and β-adrenergic responsiveness.

In yet another embodiment, the present invention results from studies designed to examine the association between specific genetic polymorphisms and depression risk after surgery (e.g., cardiac surgery). These studies demonstrate that specific genetic variants contribute to the risk of postoperative depression and suggest that monoamine oxidase A (MAOA) plays a pivotal role. It will be appreciated from a reading of this disclosure that a polymorphism in the upstream regulatory region of the gene encoding MAOA, MAOA-uVNTR, is associated with both chronic and new onset depression in women while a 44-base pair insertion/deletion polymorphism in the 5′ flanking regulatory region of the serotonin transporter gene, 5HTTLRP, is associated with chronic depression at one year but not new onset depression. New onset depression is of particular interest because it cannot be predicted pre-operatively and may be associated with CABG surgery.

Biological effects for the single nucleotide polymorphisms (SNPs) referenced above, and described in greater detail in Example 6 that follows, have been demonstrated. This embodiment of the present invention provides definitive association between these genetic variants and clinical postoperative depression in the perioperative setting. This embodiment is exemplified by reference to cardiac surgery patients but includes all perioperative, periprocedure (endoscopy, bronchoscopy, cardiac catheterization, angioplasty, etc.), and intensive care unit settings.

The presence of one or more of the above-referenced polymorphisms present in a sample (e.g., a biological sample such as blood) can be determined using any accurate detection method, including a variety of genotyping techniques known in the art (e.g., using a preoperative “CHIP” or SNP panel). Examples of such techniques include the use of polymerase chain reaction and extension primers (see too the Example below). Suitable techniques also include the use of RFLP analysis and mass spectrometry (see also Ye et al, Hum. Mutat. 17(4):305 (2001), Chen et al, Genome Res. 10:549 (2000)).

The genetic variants (SNPs) described above and in Example 6 can be used, for example, to predict postoperative and ICU depression risk. As indicated above, screening for genetic variants of the invention is also relevant for other invasive procedures including but not limited to endoscopy, bronchoscopy, cardiac catheterization, and angioplasty. Preoperative screening for genetic variants enables clinicians to better stratify a given patient for therapeutic intervention, either with drug therapy or with other modalities. Additionally, knowledge of genetic variants allows patients to choose, in a more informed way in consultation with their physician, medical versus procedural therapy. Identifying these genetic variants in patients who decide to undergo surgery or other invasive procedure enables health care providers to design altered therapeutic strategies aimed at preventing the incidence of depression in the subset of patients with enhanced risk. In addition, identifying these genetic variants in patients who have already experienced depression might also lead to alteration or modification in the therapeutic strategy.

As indicated above, preoperative genotype testing can refine risk stratification and improve patient outcome. Based on the genetic risk factors identified, treatment regimens, including drug treatment regimens, and used to minimize the risk of depression and/or associated effects can be useful in reducing depression risk in acute settings, for example, cardiac surgery. Identification of the genetic markers described herein can facilitate individually tailored medical therapy (personalized medicine) designed to reduce depression risk and associated morbidity and mortality and decreased quality of life. Recognition that a patient is at unique risk, based on genetic factors described herein, can enable early intervention in such patients i.e., immediately upon identification of depression symptoms.

In a further aspect of this embodiment, the present invention relates to methods of identifying compounds suitable for use in minimizing the risk of depression (e.g., perioperative depression, particularly new onset depression). These methods can comprise screening compounds, for example, for their ability to increase concentrations of monoamine neurotransmitters in the brain.

In a still further embodiment, the present invention results from studies designed to prospectively examine specific genetic variants involved in bleeding pathways and how they influence postoperative bleeding. A polymorphism found to be significantly associated with postoperative bleeding is the 98 G/T SNP of the E-selectin (ELAM-1) gene (rs 1805193 (www.ncbi.nlm.nih.gov/SNP/snp_ref.cgi? rs=1805193)).

Biological effects for the single nucleotide polymorphism (SNP) referenced above, and described in greater detail in Example 7, have been demonstrated. This embodiment of the present invention provides definitive association between this genetic variant and clinical postoperative bleeding in the perioperative and intensive care unit setting. This embodiment is exemplified by reference to cardiac surgery patients but includes all perioperative, periprocedure (endoscopy, bronchoscopy, cardiac catheterization, angioplasty, etc.), and intensive care unit settings.

The presence of the above-referenced polymorphism in a sample (e.g., a biological sample such as blood) can be determined using any of a variety of genotyping techniques known in the art (e.g., a “CHIP” or SNP panel). Examples of such techniques include the use of polymerase chain reaction and extension primers (see too the Example below) (Preferred primers (forward, reverse and extension) are: ACGTTGGATGCTGCTTCCCAAAACGGAAAG, ACGTTGGATGGCTGAGAGAAACTGTGAAGC and TATTTCAAGCCTAAACCTTTGGGT)). Suitable techniques also include the use of RFLP analysis and mass spectrometry (see also Ye et al, Hum. Mutat. 17(4):305 (2001), Chen et al, Genome Res. 10:549 (2000).)

The genetic variant (SNP) described above and in Example 7 can be used, for example, to predict predict postoperative and ICU bleeding. As indicated above, screening for the genetic variant of the invention is also relevant for other invasive procedures including but not limited to endoscopy, bronchoscopy, cardiac catheterization, and angioplasty. Preoperative screening for genetic variants enables clinicians to better stratify a given patient for therapeutic intervention, either with drug therapy or with other modalities to alter homeostatic pathways. Additionally, knowledge of genetic the variant allows patients to choose, in a more informed way in consultation with their physician, medical versus procedural therapy. Identifying the genetic variant in patients who are already bleeding or having difficulties with coagulation, can result in the alteration or modification of the therapeutic strategy.

As indicated above, preoperative genotype testing can refine risk stratification and improve patient outcome. Based on the genetic risk factors identified, non-specific therapies to reduce thrombin activation and bleeding, such as aprotinin (or other anti-fibrinolytic agents), prostaglandins (Kozek-Langenecker et al, Anesthesia & Analgesia 87:985-988 (1998)) or more aggressive heparin dosing (Despotis et al, Thrombosis & Haemostasis 76:902-908 (1996)), can be employed for at-risk patients.

The invention also relates to kits suitable for use in testing for the presence of the polymorphisms identified herein in connection with the various embodiments. Such kits can include, for example, reagents (e.g., probes or primers) necessary to identify the presence of the above-referenced polymorphisms. The invention also relates to probes or primers suitable for use in identifying the presence of the above-referenced polymorphisms bound to a solid support.

In addition to perioperative and periprocedure settings, the screening procedures described herein can also be important for predicting medical response to a variety of extremely stressful situations, including (but not limited to) accidents (e.g., automobile, etc.) that can require subsequent emergency room evaluation and/or hospitalization, as well as combat and terrorism casualties.

It is known that genetic variants often travel in groups along a chromosome (haplotype blocks). Thus, identification of the above-referenced SNPs as being associated with perioperative risk of myocardial injury, MACE, cognitive decline, arrhythmias, depression or bleeding may uniquely identify additional SNP(s) present in such a haplotype block. While a SNP associated with a disease/disorder may not be the definitive causative SNP producing the effect, it is nonetheless a valid marker, often in linkage disequilibrium, or directly in a haplotype block, with the causative SNP. Resequencing of the genomic DNA region around the SNPs specifically identified herein may reveal other SNP(s) associated with perioperative risk of myocardial injury, MACE, cognitive decline, arrhythmias, depression or bleeding (i.e., the associated interval)—these could be predicted based upon their presence in the same haplotype or by being in linkage disequilibrium with the specific SNPs disclosed herein. The foregoing applies equally to SNPs disclosed in U.S. application No. Ser. 10/979,816, which is equivalent to WO 2005/041896 (the disclosures of which are incorporated herein by reference), and thus while the SNPS specified in application Ser. No. 10/979,816 are not the subject of the present invention, the instant invention does relate to other SNPs within the haplotype blocks as those specified in application Ser. No. 10/979,816, or SNPs in linkage disequilibrium therewith.

Certain aspects of the above described embodiments of the invention are described in greater detail in the non-limiting Examples that follows (see also U.S. Pat. No. 5,418,162, Lesch et al, Science 274:1527-1531 (1996) and U.S. Provisional Application No. 60/516,313).

### Example 1

### Experimental Details

Study Population

The patients enrolled in this study were part of the Perioperative Genetics and Safety Outcomes Study (PEGASUS), an ongoing Institutional Review Board approved, prospective, longitudinal study at Duke University Medical Center. The current substudy targets a cohort of patients undergoing elective cardiac surgery utilizing cardiopulmonary bypass (CPB), during a specified period, in whom serial postoperative serum markers of myocardial injury were measured. Patients were excluded if they had a history of symptomatic cerebrovascular disease, renal failure, active liver disease, or bleeding disorders.

Patient Management

Anesthesia was induced and maintained with midazolam, fentanyl, and isoflurane with muscle relaxation provided by pancuronium. All patients underwent nonpulsatile hypothermic (30°-32° C.) cardiopulmonary bypass. The perfusion apparatus consisted of the Cobe CML membrane oxygenator™ (COBE Chem Labs, Lakewood, Colo.), the Sarns 7000 MDX pump™ (3M Inc, Ann Arbor, Mich.), and the Pall SP3840™ arterial line filter (Pall Biomedical Products Co, Glen Cove, N.Y.). Perfusion was maintained at pump flow rates of 2 to 2.4 L·min⁻¹·m² throughout CPB. The pump was primed with crystalloid, and serial hematocrits were kept at ≧0.18 with packed red blood cell transfusion as necessary. Arterial blood gases were followed every 15 to 30 minutes to maintain arterial carbon dioxide partial pressures at 35 to 40 mm Hg, unadjusted for temperature (a-stat), and oxygen partial pressures at 150 to 250 mm Hg.

Definition of Myocardial Injury Phenotype

In 432 patients, serum was collected for measurement of creatine kinase-MB isoenzyme (CK-MB) levels at the following time points: prior to induction of anesthesia (baseline), upon aortic cross-clamp release, and at 4.5, 24 and 48 hours after aortic cross-clamp removal. Collected blood was centrifuged and the resultant supernatant was immediately frozen at −70° C. until analysis was completed. Immunoassays were forward immunometric (sandwich) assays performed by Biosite Diagnostics (San Diego, Calif.) in a 10-μl reaction volume in 384-well microtiter plates using a Tecan Genesis RSP 200/8 Workstation (Tecan, Research Triangle Park, N.C.). In addition to test samples, each plate contained a calibration curve consisting of multiple analyte concentrations and control samples. The plates were read by a fluorometer (Tecan Spectraflur) with an excitation wavelength of 430 nm and an emission wavelength of 570 nm. Each well was read six times at 114-s intervals, and a rate of fluorescence generation was calculated. Calibration curves were eight points tested at multiple locations on the assay plate. The calibration curve was calculated using a five-parameter logistic fit and sample concentration was determined. The upper limit of normal for CK-MB values for this laboratory is 5 ng/ml.

Postoperative myocardial injury was defined as a CK-MB level ≧50 ng/ml (i.e. 10 times the upper limit of normal for the reference laboratory) at 24 hours postoperatively.

Candidate Polymorphisms Selection

Twenty three candidate genes involved in the pathogenesis of myocardial ischemia-reperfusion injury were selected a priori based on previous gene expression results in animal models, pathway analysis, a review of linkage and association studies reported in the literature, and expert opinion. Forty eight single nucleotide polymorphisms (SNPs) were subsequently selected based on literature review and predictive analyses (SIFT—Sorting Intolerant from Tolerant) and tested for association with postoperative myocardial injury (Table 1).

Isolation of Genomic DNA and Genotype Analysis

Whole blood was collected preoperatively with genomic DNA extraction subsequently performed using the Puregene™ system (Gentra Systems, Minneapolis, Minn.). Genotyping assays were conducted at Agencourt Bioscience Corporation (Beverly, Mass.) by Matrix Assisted Laser Desorption/Ionization Time-Of-Flight (MALDI-TOF) mass spectrometry, using the Sequenom MassArray™ system (Sequenom, San Diego, Calif.) (Sun et al, Nucleic Acids Res. 28(12):E68 (2000)). Primers used and polymorphism details can be found at anesthesia.duhs.duke.edu/pegasus/myocardinj/1/website table 1.htm. Genotype accuracy of the Sequenom MassArray™ system was estimated at 99.6% (Gabriel et al, Science 296(5576):2225-2229 (2002)). Using direct sequencing on an AB13700 capillary sequencer (Applied Biosystems, Foster City, Calif.), genotyping reproducibility in this study was validated to be >99% by scoring a panel of 6 polymorphisms in 100 randomly selected patients.

Statistical Analysis

To test for association between the 48 candidate gene polymorphisms and incidence of postoperative myocardial injury a two-stage analysis approach was employed: marker selection, followed by model building (Hoh et al, Ann. Hum. Genet. 64(PT5):413-417 (2000)).

First, descriptive statistics, including allele and genotype frequencies were calculated for each polymorphism, and Hardy-Weinberg equilibrium was evaluated using an exact test among controls (Guo et al, Biometrics 48(2):361-372 (1992)). To avoid assumptions regarding the modes of inheritance, all analyses were performed using additive (homozygote major allele versus heterozygote versus homozygote minor allele), dominant (homozygote major allele versus heterozygote+homozygote minor allele), or recessive (homozygote major allele+heterozygote versus homozygote minor allele) models for each polymorphism. The genotype frequencies between case and control patients were then compared using univariate Chi square test for each of the 48 polymorphisms, and selected a set of influential markers was selected based on a nominal p-value of less than 0.1.

Second, a series of logistic regression analyses were performed to test the independent main effects of all pairs of markers selected in the previous step on incidence of postoperative myocardial injury (2-SNP main effects models). After adjusting for multiple testing, the subgroup of SNPs present in the significant 2-SNP main effects models were evaluated in logistic regression models containing groups of 3 SNPs (3-SNP main effects models); these models were developed by sequentially adding one SNP at a time to the 2-SNP main effects models. Similarly, the SNPs present in the significant 3-SNP main effect models were used to develop 4-SNP main effects models of myocardial injury.

One criticism of genetic association studies is that population stratification can result in false-positive results. To account for this possibility, self-reported ethnicity (white versus non-white) was included as a covariate in multiple logistic regression models. Similarly, procedure type and gender were tested as covariates in multiple logistic regression models incorporating the genetic information.

Because the analysis strategy employed many separate tests of independence, an adjustment of observed p-values was required to account for this multiple testing. Random permutation analysis was used to adjust p-values from the logistic regressions. Four thousand copies of the data set were generated, randomly reassigning myocardial injury to study subjects, thereby dissociating genotype from phenotype in the copies. For each permutation, p-values were calculated from logistic models with each pair of SNPs. From these SNP pair models the smallest p-value was retained to estimate the distribution of 4000 “smallest” p-values under the null hypothesis of no association. An adjusted p-value was computed as the fraction of permutation p-values that were smaller than the observed p-value. For example, if among 4000 permutations, 40 p-values were smaller than the observed p-value, then the adjusted p-value would be 40/4000 or 0.01.

All statistical analysis was performed using SAS and SAS/Genetics version 8.02 (SAS Inc, Cary, N.C.). Predictive analyses to identify amino-acid substitutions that affect protein function were performed using the SIFT (Sorting Intolerant from Tolerant) program available at blocks.fhcrc.org/sift/SIFT.html. Continuous variables were described as mean±standard deviation; categorical variables were described as percentages. Adjusted p-values of <0.05 (Bonferroni correction or permutation testing) were considered significant.

### Results

The incidence of perioperative myocardial injury in the study population was 12% (52/432). The minor allele frequencies for the 48 polymorphisms examined among the unaffected patients (no postoperative myocardial injury) are presented in Table 1. Four polymorphisms had deviations from Hardy-Weinberg equilibrium in both the white unaffected and myocardial injury groups, and were excluded from subsequent analyses. Based on the first step of selection process, a set of 11 influential markers were selected for modeling, identified in Table 1 in bold/italics/blue.

TABLE 1
Single nucleotide polymorphisms evaluated for their relationship
to postoperative myocardial injury after cardiac surgery.

| | | Nucleotide | Cytogenetic | |
|---|---|---|---|---|
| SNPid* | Gene | Substitution | Locus | Functio [symbol] |
| rs1800629 | TNFA (Tumor necrosis alpha) | −308G/A | 6p21.3 | cytokines/ |
| rs361525 | TNFA | −238G/A | 6p21.3 | inflammation |
| rs1800610 | TNFA | +488G/A | 6p21.3 | |
| [symbol] | [symbol] | [symbol] | 2q14 | |
| rs1143633 | IL1B | 5810G/A | 2q14 | |
| [symbol] | [symbol] | [symbol] | 2q14 | |
| rs1800587 | IL 1A (Interleukin 1 alpha) | −889 C/T | 2q14 | |
| rs1799942 | IL 1A | 4844 G/T | 2q14 | |
| rs2229235 | IL1RN (IL1-Ra; Interleukin1-receptor antagonist) | 8006T/C | 2q14.2 | |
| rs2229234 | IL1RN | 11100T/C | 2q14.2 | |
| rs1800795 | IL6 (Interleukin 6) | −174G/C | 7p21 | |
| [symbol] | [symbol] | [symbol] | 7p21 | |
| [symbol] | [symbol] | [symbol] | 7p21 | |
| rs4073 | IL8 (Interleukin 8) | −251A/T | 4q12-q13 | |
| rs3021097 | IL10 (Interleukin 10) | −819C/T | 1q31-q32 | |
| rs1800872 | IL10 | −592C/A | 1q31-q32 | |
| rs1800947 | CRP (C reactive protein) | 1059G/C | 1q21-q23 | |
| [symbol] | [symbol] | [symbol] | 1q21-q23 | |
| [symbol] | [symbol] | [symbol] | 1q23-q25 | leukocyte- |
| rs5355 | ELAM1 | 1839C/T | 1q23-q25 | endothelium |
| [symbol] | [symbol] | [symbol] | 1q23-q25 | adhesion |
| rs6131 | SELP (P-selectin) | 1087G/A | 1q23-q25 | |
| rs6127 | SELP | 1902A/G | 1q23-q25 | |
| rs6133 | SELP | 2013G/T | 1q23-q25 | |
| rs6136 | SELP | 2361A/C | 1q23-q25 | |
| rs1800805 | SELP | −1969A/G | 1q23-q25 | |
| rs1799969 | ICAMI (Intercellular Adhesion Molecule 1) | 778G/A | 19p13.3-p13.2 | |
| [symbol] | [symbol] | [symbol] | 19p13.3-p13.2 | |
| rs1801714 | ICAM1 | 1112C/T | 19p13.3-p13.2 | |
| rs1800821 | VCAM1 (Vascular Cell Adhesion Molecule 1) | −1594T/C | 1p32-p31 | |
| rs12953 | PECAM1(Platelet-Endothelial Cell Adhesion Molecule 1) | 1688G/A | 17q23 | |
| rs1042579 | THBD (Thrombomodulin) | 1959C/T | 20p11.2 | |
| SNP014** | CTSG (Cathepsin G) | 2108A/G | 14q11.2 | leukocyte pr [symbol] |
| rs1341023 | BPI (Bactericidal/Permeability-Increasing Protein) | 270T/C | 20q11.23-q12 | endotoxin |
| [symbol] | [symbol] | [symbol] | 2q11.23-q12 | responsiveness |
| rs2232618 | LBP | 42711T/C | 2q11.23-q12 | |
| [symbol] | [symbol] | [symbol] | 5q31.1 | |
| SNP007** | TLR4 (Toll-Like Receptor 4) | 896A/G | 9q32-q33 | |
| SNP008 | TLR4 | 1196C/T | 9q32-q33 | |
| rs2070744 | NOS3 (ecNOS, Endothelial Nitric Oxide Synthase) | −786T/C | 7q36 | free |
| rs1799983 | NOS3 | 894G/T | 7q36 | radical |
| rs1799985 | NOS3 | 10(in23)G/T | 7q36 | injury |
| rs1799895 | SOD3 (EC-SOD, Superoxide Dismutase 3) | 760C/G | 4pter-q21 | |
| [symbol] | [symbol] | [symbol] | 11p13 | |
| rs1001179 | CAT | −262C/T | 11p13 | |
| SNP009** | CAT | −21A/T | 11p13 | |
| rs2075800 | HSPA1L (HSP70-hom, Heat Shock Protein 70-homologous) | 1804G/A | 6p21.3 | heat shock res [symbol] |
| rs2227956 | HSPA1L | 1478C/T | 6p21.3 | |

*From NCBI's dbSNP public database (www.ncbi.nlm.nih.gov/SNP/)
**Duke polymorphism ID number
Note:
The set of 11 influential markers selected in the first-stage of analysis is presented in bold/italics/blue. [symbol]
indicates data missing or illegible when filed

Logistic regression models evaluated all possible pairwise combinations of these markers (2-SNP main effects models), 9 of which remained significant after a Bonferroni correction for multiple testing. These 9 models were used to build 3-SNP main effects logistic regression models, 6 of which remained significant after Bonferroni correction. Finally, these 6 models were used to build 4-SNP main effects models, 4 of which remained significant after Bonferroni correction. The final 4-SNP main effects models included combinations of 6 SNPs: CRP 1846C/T, LBP 19983T/C, IL6-572G/C, ELAM1 98G/T, CAT -844C/T, and ICAM1 1462A/G. Presence of the minor allele at four of these loci had a deleterious effect (CRP 1846T OR 2.4 [95% CI 1.1-5.1]; LBP 19983C OR 2.5 [1.2-4.8]; IL6-572C OR 2.4 [1-5.9]; ICAM1 1462G), and was protective at the other two loci (ELAM1 98T OR 0.1[0.02-0.9]; CAT-844T OR 0.5[0.3-0.8]). Collectively, these 6 SNPs result in models with receiver operator characteristic curves (c-indices) ranging from 0.72 to 0.75. In a multiple logistic regression model, no evidence was found for an interaction between these genetic polymorphisms and procedure type, gender or race in explaining the incidence of postoperative myocardial injury (Table 2).

TABLE 2
Results of multiple logistic regression models testing the independent
effects of combinations of 4 influential markers (4-SNP main effects)
on incidence of postoperative myocardial injury.

| | | Genetic | Genetic | | |
|---|---|---|---|---|---|
| Predictor SNPid | SNP | Model | Model | | Covariate |
| (4-SNP models) | p-value | p-value | C-index | Covariates | p-value |
| rs1805193 | 0.03 | 7.04e−6 | 0.75 | Procedure Type* | 0.59 |
| rs1205 | 0.01 | | | Gender | 0.34 |
| rs2232582 | 0.009 | | | Race** | 0.6 |
| rs769214 | 0.01 | | | | |
| rs1805193 | 0.02 | 9.23e−6 | 0.73 | Procedure Type | 0.46 |
| rs1205 | 0.05 | | | Gender | 0.65 |
| rs1800796 | 0.007 | | | Race | 0.78 |
| rs769214 | 0.01 | | | | |
| rs1805193 | 0.02 | 2e−5 | 0.72 | Procedure Type | 0.49 |
| rs1135683 | 0.01 | | | Gender | 0.92 |
| rs1800796 | 0.008 | | | Race | 0.23 |
| rs769214 | 0.03 | | | | |
| rs1805193 | 0.04 | 3e−5 | 0.73 | Procedure Type | 0.17 |
| rs1205 | 0.02 | | | Gender | 0.34 |
| rs2232582 | 0.02 | | | Race | 0.79 |
| rs1135683 | 0.04 | | | | |

SNP—single nucleotide polymorpshism; C-index - area under the receiver operator characteristic curve;
*coronary artery bypass grafting (CABG) versus valve procedure;
**white versus non-white

### Example 2

### Experimental Details

### Study Design and Population:

Prospective cohort study of 434 patients (371 white, 63 non-white) undergoing cardiac surgery with CPB.

### Phenotype Definition:

Serial determinations of serum creatine kinase-MB isoenzyme (CK-MB) mass levels were performed using an immunometric (sandwich) assay (Biosite Diagnostics, San Diego, Calif.) with an upper limit of normal of 5 ng/ml. Postoperative myocardial injury defined as a CK-MB level ≧50 ng/ml (i.e. 10 times upper limit of normal for the reference laboratory) at 24 hours postoperatively (Newby et al, Am. J. Cardiol. 144:957 (2002)). Patients with abnormal baseline CK-MK levels were excluded.

### Selection of Candidate Gene Polymorphisms:

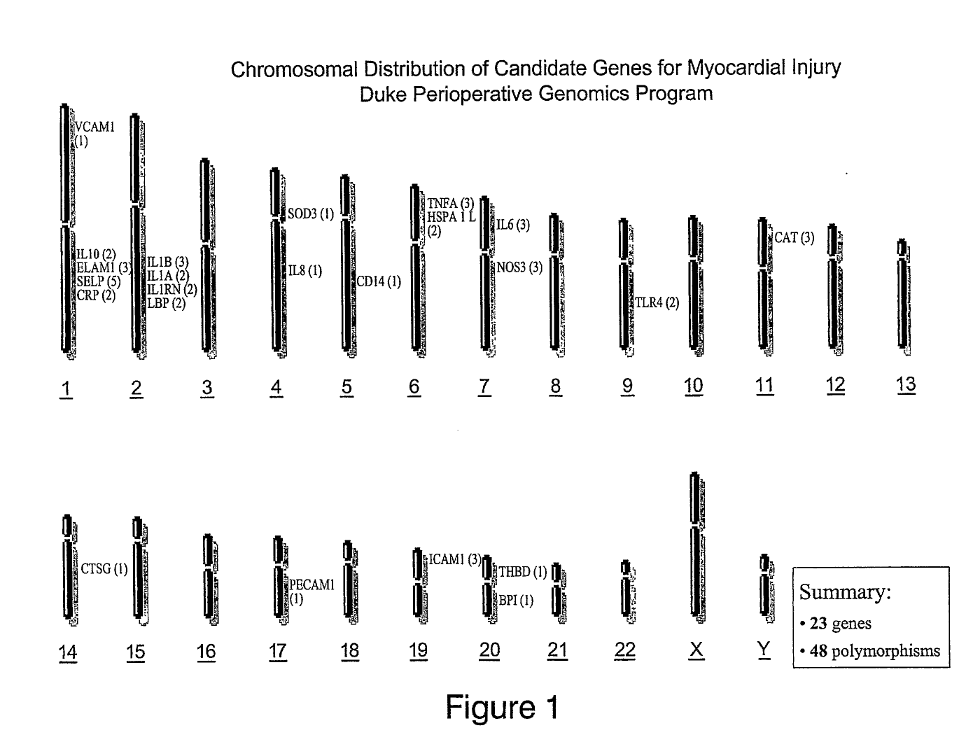

23 candidate genes involved in the pathogenesis of inflammation and myocardial ischemia/reperfusion injury were selected a priori based on previous transcription profiling in animals models, pathway analysis, a review of linkage and association studies reported in the literature, and expert opinion (FIG. 1). 48 SNPs were subsequently selected in these process-specific candidate genes, based on literature review and predictive analyses (SIFT-Sorting Intolerant program) Ng et al, Nuc. Acids Res. 31:3812 (2003)), with an emphasis on functionally important variants.

### Genotyping:

MALDI-TOF mass spectrometry (Sequence massARRAY).

### Genetic Marker Analysis:

Descriptive statistics (allele and genotype frequencies) and Hardy-Weinberg equilibrium (HWE) were evaluated for each SNP. All analyses were performed using dominant, recessive and additive modes of inheritance. A two-stage analysis was then performed: (1) marker selection, and (2) modeling of gene-phenotype effects (Hoh et al, Ann. Hum. Genet. 64:413 (2000)). An influential subset of markers was first selected using univariate Chi-square tests with a nominal p-value <0.1. Second, a series of logistic regression models were used to test sequentially the independent mail effects of groups of 2, 3, and 4 pre-selected SNPs on incidence of postoperative myocardial injury. Adjustment for multiple testing was performed by Bonferroni correction and permutation testing (4000 replicates) (Good PI. Permutation tests: a practical guide to resampling methods for testing hypotheses, 2^{nd} ed., 2000). Self-reported ethnicity (Tang et al, Am. J. Hum. Genet. 76:268-75 (2005), procedure type and gender were tested as covariates in multiple logistic regression models incorporating genetic information. All statistical analyses performed using SAS/Genetics v8.02 (SAS Inc, Cary, N.C.); SNP predictive analyses performed using SIFT program (blocks.fhcrc.org/sift/SIFT.html).

### Results

The incidence of postoperative myocardial injury in the patient population was 12% (52/434). Demographic and procedural characteristics were not different between affected and unaffected groups.

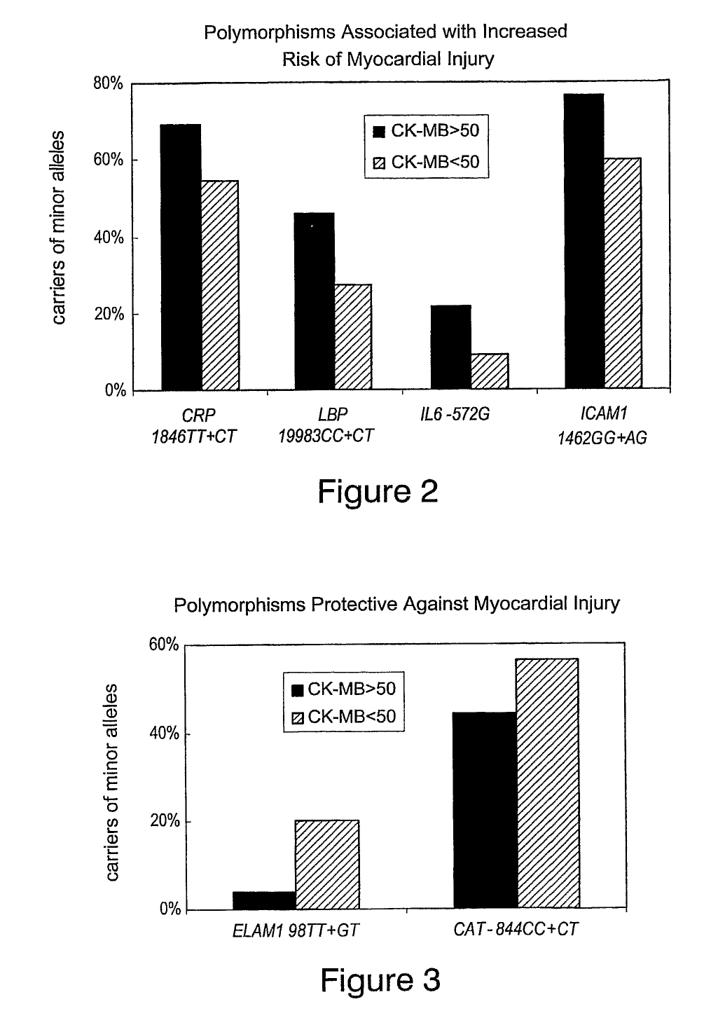

Two SNPs had deviations from HWE and were excluded from subsequent analyses. Based on the first-stage analyses, a set of 11 markers were selected for modeling. 9 logistic regression models evaluating 2-SNP main effects were significant after Bonferroni correction, and were used to develop 3-SNP models, 6 of which were significant after Bonferroni correction. Finally, 4-SNP models were developed and 4 remained significant after Bonferroni correction. These included combinations of 6 SNPs, identified in Table 3. Presence of minor allele at four of these loci had a deleterious effect (FIG. 2), and was protective at the other two loci (FIG. 3).

TABLE 3
Genotype Frequencies for Polymorphisms Associated
with Incidence of Postoperative Myocardial Injury

| SNPid | CK-MB>50 | | CK-MB<50 | | OR(D) [symbol] | 95% CI |
|---|---|---|---|---|---|---|
| rs1205 | C/C | 0.306 | C/C | 0.455 | 2.4 | 1.1-5.1 |
| CRP1846C/T | C/T | 0.633 | C/T | 0.467 | | |
| | T/T | 0.061 | T/T | 0.078 | | |
| | T/T | 0.542 | T/T | 0.729 | | |
| rs2232582 | C/T | 0.417 | C/T | 0.239 | 2.5 | 1.2-4.8 |
| LBP19983T/C | C/C | 0.042 | C/C | 0.032 | | |
| rs1800796 | G/G | 0.784 | G/G | 0.911 | 2.4 | 1-5.9 |
| IL6-572G/C | C/G | 0.216 | C/G | 0.089 | | |
| rs1135683 | A/A | 0.235 | A/A | 0.405 | 1.79 | 1.14- |
| ICAM1 | A/G | 0.490 | A/G | 0.464 | | 2.82 |
| 1462A/G | G/G | 0.275 | G/G | 0.131 | | |
| | G/G | 0.959 | G/G | 0.799 | | |
| rs1805193 | G/T | 0.020 | G/T | 0.184 | 0.1 | 0.02-0.9 |
| ELAM1 98G/T | T/T | 0.020 | T/T | 0.017 | | |
| | T/T | 0.558 | T/T | 0.437 | | |
| rs769214 | C/T | 0.365 | C/T | 0.429 | 0.5 | 0.3-0.8 |
| CAT-844T/C | C/C | 0.077 | C/C | 0.134 | | |

[symbol]

indicates data missing or illegible when filed

Collectively, these 6 SNPs result in models with area under the receiver operator characteristic vurves (c-index) of 0.72-0.75. No evidence was found for an interaction between these genetic polymorphisms and procedure type, gender, and race in explaining the incidence of postoperative myocardial injury (see Table 4).

Results of Multiple Logistic Regression Models

| | | Genetic | Genetic | | |
|---|---|---|---|---|---|
| Predictor SNPid | SNP | Model | Model | | Covariate |
| (4-SNP models) | p-value^{#} | p-value^{##} | C-index | Covariates | p-value |
| rs1805193 | 0.03 | 3.5e−5 | 0.75 | Procedure Type* | 0.59 |
| rs1205 | 0.01 | | | Gender | 0.34 |
| rs2232582 | 0.009 | | | Race** | 0.6 |
| rs769214 | 0.01 | | | | |
| rs1805193 | 0.02 | 4.6e−5 | 0.73 | Procedure Type | 0.46 |
| rs1205 | 0.05 | | | Gender | 0.65 |
| rs1800796 | 0.007 | | | Race | 0.78 |
| rs769214 | 0.01 | | | | |
| rs1805193 | 0.02 | 1e−4 | 0.72 | Procedure Type | 0.49 |
| rs1135683 | 0.01 | | | Gender | 0.92 |
| rs1800796 | 0.008 | | | Race | 0.23 |
| rs769214 | 0.03 | | | | |
| rs1805193 | 0.04 | 1.5e−4 | 0.73 | Procedure Type | 0.17 |
| rs1205 | 0.02 | | | Gender | 0.34 |
| rs2232582 | 0.02 | | | Race | 0.79 |
| rs1135683 | 0.04 | | | | |

^{#}Permutation-adjusted p-value;
^{##}Bonferroni-adjusted p-value;
*CABG versus valve;
**whites versus non-whites

In conclusion, functional genetic variants in cytokine, leukocyte-endothelial interaction, endotoxin responsiveness, and free radical injury pathways are independent risk factors modulating the severity of myocardial injury after cardiac surgery. This may aid in preoperative identification of high-risk cardiac surgical patients and the development and testing of novel cardioprotective strategies in genotype-stratified patient populations.

### Example 3

### Experimental Details

### Patient Sample:

Patients undergoing CABG surgery with cardiopulmonary bypass (CPB) with genotype data were included in the analysis sample. Patients were stratified into two subgroups based on the type of surgery performed: isolated CABG or CABG with any type of valve surgery.

### Definition of Endpoint:

A composite endpoint, major adverse cardiac event (MACE), was defined for each patient. Relevant events included death, MI, repeat CABG, repeat revascularization, or cardiac arrest. For each patient, time (number of days) to the first of these events to occur following surgery was considered as the outcome in a survival analysis.

### Statistical Analysis:

17 candidate gene polymorphisms were selected for analysis based on published associations with cardiovascular outcomes (Table 5). Polymorphisms were examined for both main effects and gender interaction associations with MACE. SNPs were characterized in three ways for the purposes of statistical analysis: 1) the absence of presence of the minor allele 2) the absence of presence of the major allele and 3) the number of minor alleles (0,1 or 2). Each characterization of each SNP was investigated with a log-rank test for association with time to event. Simple main effects of each SNP, gender, and procedure type were investigated, as well as SNP by gender interactions. Patients who did not experience an event or who were lost to follow-up were censored at time of last contact.

TABLE 5
List of Candidate Genes and their Polymorphisms

| Candidate | | | |
|---|---|---|---|
| Gene | Gene Function | Polymorphism | SNP ID |
| Integrin, beta | mediates the final common | PI^{A2} allele = human platelet antigen-1b | rs5918 |
| 3 or Platelet | pathway in the formation of | (HPA-1b or Pl^{A2}) = sequence variation in | |
| Glycoprotein | thrombosis; GPIIIa and GPIIb | exon 2, leading to substitution of leucine | |
| IIIa (platelet | form the fibrinogen and von | (Pl^{A1}) for proline (Pl^{A2}) at position 33 | |
| GPIIIa) | Willebrand factor receptor on | (1565T/C) | |
| (ITGB3) | the surface of platelets | | |
| Interleukin 6, | IL6 is an immunoregulatory | −572G/C | rs1800796 |
| (IL6) | cytokine that activates a cell- | −174G/C | rs1800795 |
| | surface signaling assembly | −597G/A | rs1800797 |
| | composed of IL6, IL6RA, and | | |
| | the shared signaling receptor | | |
| | gp130 | | |
| C-reactive | acute phase reactant; levels are | 1059G/C | rs1800947 |
| protein (CRP) | predictive of cardiovascular | 3′FI T/C (1846C/T) | rs1205 |
| | events in patients with CAD | | |
| Tumor | Inflammatory cytokine, affects | −308G/A | rs1800629 |
| Necrosis Alpha | lipid metabolism and predispose | −238G/A | rs361525 |
| (TNFA) | to obesity related insulin | | |
| | resistance | | |
| Plasminogen- | | −675indel(5G/4G) | rs1799768 |
| Activator | | −844A/G | rs2227631 |
| Inhibitor type 1 | | | |
| (PAI1) | | | |
| Selectin P | adhesion molecule, expressed at | T715P = 2361A/C = SELP_1 | rs6136 |
| (SELP) | the surface of activated cells, | S290N = 1087G/A = SELP_3 | rs6131 |
| | that mediates the interaction of | N562D = 1902A/G = SELP_4 | rs6127 |
| | activated endothelial cells or | | |
| | platelets with leukocytes; CD24 | | |
| | is a ligand for P-selectin | | |
| Angiotensin I | hydrolyzing angiotensin I into | (in16)indel (W/-) = ACE/ID | SNP030 |
| converting | angiotensin II, a potent | | |
| enzyme (ACE) | vasopressor, and aldosterone- | | |
| | stimulating peptide | | |
| β2-adreno- | | R16G = 46A/G | rs1042713 |
| ceptor(ADRB2) | | Q27E = 79C/G | rs1042714 |
| Matrix | Matrix metalloproteinases | −11715A/6A | rs3025058 |
| metalloproteinase | (MMPs) may contribute to | | |
| 3 (MMP3) | weakening of the cap, which | | |
| | favors rupture. Stromelysin, a | | |
| | member of MMP family, is | | |
| | identified extensively in human | | |
| | coronary atherosclerotic lesions. | | |

Further investigation with Cox proportional hazards regression analysis allowed adjustment for preoperative gender differences. Covariates included in each model were Charlson Comorbidity score and patient age. All analyses were conducted with SAS version 8.02. P<0.05 was considered significant.

### Results:

Gender differences in demographic, clinical and procedural variables in the groups of patients undergoing isolated CABG surgery (IC) and combined CABG-valve surgery (CV) are presented in Tables 6 and 7. Patients undergoing IC developed fewer major adverse cardiac events during the entire period of follow up as compared to the CV group (20.8% and 26.3%, respectively).

TABLE 6
Demographic, clinical and procedural variables by
gender for the population of patients undergoing
isolated CABG surgery (N = 1753)

| | Males | Females | P value |
|---|---|---|---|
| Demographics | | | |
| Age (years) | 62.28 (10.69) | 65.32 (10.63) | <.0001 |
| Race (% white) | 86.51 | 73.71 | <.0001 |
| Body Mass Index | 29.78 (14.76) | 30.12 (13.30) | .64 |
| (kg/m²) | | | |
| Body Surface Area | 2.05 (0.19) | 1.80 (0.20) | <.0001 |
| (m²) | | | |
| Preoperative Meds | | | |
| Beta blockers (%) | 71.96 | 74.22 | .34 |
| Past Medical History | | | |
| Diabetes (%) | 29.41 | 42.25 | <.0001 |
| Hypertension (%) | 64.46 | 77.71 | <.0001 |
| Renal Dialysis (%) | 0.80 | 1.63 | .48 |
| Prior MI (%) | 32.11 | 21.23 | <.0001 |
| CHF (%) | 13.26 | 18.99 | .002 |
| Redo Surgery (%) | 5.07 | 3.31 | .11 |
| Prior PTCA (%) | 13.59 | 13.95 | .89 |
| Ejection Fraction | 51.21 (13.56) | 53.18 (13.18) | .01 |
| Intra-operative Data | | | |
| Cross-clamp time | 62.07 (27.43) | 58.77 (27.50) | .02 |
| (min) | | | |
| Bypass time (min) | 112.57 (41.89) | 108.26 (43.91) | .05 |

CABG—coronary artery bypass grafting, MI—myocardial infarction, CHF—congestive heart failure, PTCA—percutaneous transluminal coronary angioplasty
Values are means (and standard deviations) or percentages. Comparisons are made with t-tests for continuous variables, and with chi-square tests for categorical variables.

TABLE 7
Demographic, clinical and procedural variables by gender
for the population of patients undergoing combined CABG
and valvular surgery (N = 190)

| | Males | Females | P value |
|---|---|---|---|
| Demographics | | | |
| Age (years) | 68.15 (10.64) | 69.33 (10.86) | .45 |
| Race (% white) | 86.84 | 88.16 | .79 |
| Body Mass Index | 18.15 (5.04) | 27.86 (8.47) | .79 |
| (kg/m²) | | | |
| Body Surface Area | 2.03 (0.20) | 1.74 (0.24) | <.0001 |
| (m²) | | | |
| Preoperative Meds | | | |
| Beta blockers (%) | 60.71 | 54.93 | .44 |
| Past Medical History | | | |
| Diabetes (%) | 22.12 | 32.39 | .12 |
| Hypertension (%) | 75.22 | 66.20 | .19 |
| Renal Dialysis (%) | 7.89 | 3.85 | .35 |
| Prior MI (%) | 12.16 | 7.69 | .42 |
| CHF (%) | 66.37 | 76.06 | .16 |
| Redo Surgery (%) | 20.18 | 18.42 | .76 |
| Prior PTCA (%) | 3.67 | 8.57 | .16 |
| Ejection Fraction | 43.00 (15.57) | 48.51 (13.58) | .02 |
| Intra-operative Data | | | |
| Cross-clamp time | 127.29 (50.10) | 124.03 (56.85) | .69 |
| (min) | | | |
| Bypass time (min) | 204.75 (73.13) | 197.32 (72.23) | .49 |

CABG—coronary artery bypass grafting, MI—myocardial infarction, CHF—congestive heart failure, PTCA—percutaneous transluminal coronary angioplasty
Values are means (and standard deviations) or percentages. Comparisons are made with t-tests for continuous variables, and with chi-square tests for categorical variables.

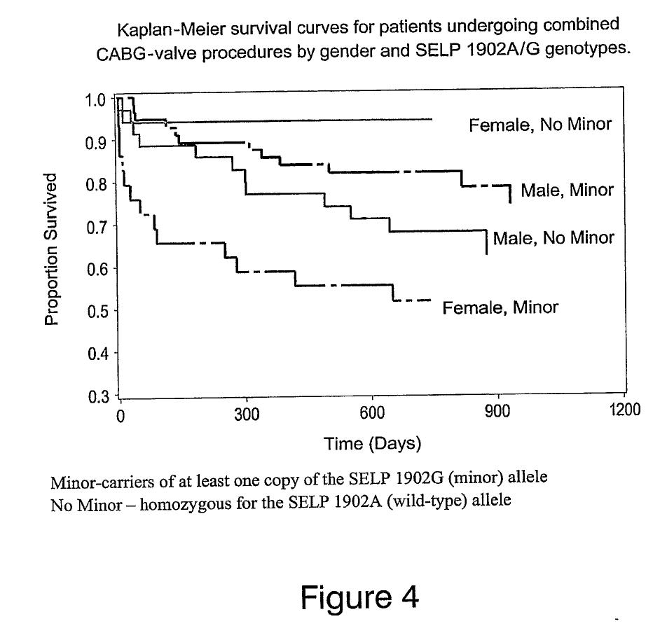

None of the candidate SNPs exhibited any significant association with MACE in the IC group (Table 8). However, in the CV group, a highly significant interaction between gender and the SELP 1902A/G (rs6127) polymorphism was found (p<0.001, Table 9). As shown with Kaplan-Meier curves in FIG. 4, female carriers of the minor allele experienced significantly more MACE when compared to other groups (hazard ratio of 18.5; 95% CI: 3.4-99.7). This significant finding persisted in multivariable regression modeling adjusting for age or Charlson comorbidity score (p<0.001).

Results of tests for association between gender-SNP interactions
and MACE in the isolated CABG (IC) population

| SNPID | pvalue | HazardRatio | HRLowerCL | HRUpperCL | Total | pctevents | GENE | ALLELE |
|---|---|---|---|---|---|---|---|---|
| RS3025058 | 0.155 | 0.701 | 0.429 | 1.143 | 1423 | 21.0822 | MMP3 | −1171indel(5A/6A) |
| RS1800629 | 0.176 | 0.711 | 0.434 | 1.165 | 1671 | 20.8857 | TNFA | −308G/A |
| SNP030 | 0.331 | 0.790 | 0.491 | 1.271 | 1626 | 20.7257 | ACE | (in16)indel(W/-) |
| RS1205 | 0.346 | 1.244 | 0.791 | 1.956 | 1566 | 20.6258 | CRP | 3′FI T/C |
| RS6127 | 0.421 | 0.820 | 0.506 | 1.329 | 1461 | 20.4654 | SELP | 1902A/G |
| RS2227631 | 0.492 | 0.846 | 0.525 | 1.364 | 1682 | 20.8680 | PAI-1 | −844A/G |
| RS5918 | 0.569 | 1.745 | 0.718 | 2.825 | 1618 | 21.0135 | ITGB3 | 1565T/C |
| RS1800796 | 0.606 | 0.835 | 0.420 | 1.658 | 1699 | 20.6592 | IL6 | −572G/C |
| RS1800797 | 0.695 | 0.909 | 0.564 | 1.465 | 1584 | 20.8965 | IL6 | −597G/A |
| RS1800795 | 0.760 | 0.926 | 0.666 | 1.517 | 1510 | 21.1921 | IL6 | −174G/C |
| RS1799768 | 0.764 | 0.929 | 0.574 | 1.503 | 1714 | 20.8285 | PAI-1 | −675indel(5G/4G) |
| RS6131 | 0.789 | 0.937 | 0.583 | 1.508 | 1621 | 20.8513 | SELP | 1087G/A |
| RS1042714 | 0.793 | 1.067 | 0.656 | 1.736 | 1528 | 20.0916 | ADRB2 | 79C/G |
| RS6136 | 0.829 | 1.066 | 0.596 | 1.909 | 1643 | 20.3895 | SELP | 2361A/C |
| RS1800947 | 0.976 | 1.009 | 0.546 | 1.866 | 1679 | 20.7266 | GRP | 1059G/C |
| RS361525 | 0.992 | 0.996 | 0.469 | 2.114 | 1606 | 20.9838 | TNFA | −238G/A |
| RS1042715 | 0.993 | 0.998 | 0.620 | 1.607 | 1544 | 20.4016 | ADRB2 | 46A/G |

SNP—single nucleotide polymorphism
MACE—major adverse cardiac events
CABG—coronary artery bypass grafting

Results of tests for association between gender-SNP interactions
and MACE in the combined CABG-valve (CV) population

| SNPID | pvalue | HazardRatio | HRLowerCL | HRUpperCL | Total | pctevents | GENE | ALLELE |
|---|---|---|---|---|---|---|---|---|
| RS6127 | 0.001 | 18.526 | 3.437 | 99.849 | 154 | 25.9740 | SELP | 1902A/G |
| RS6131 | 0.180 | 0.432 | 0.127 | 1.471 | 180 | 26.1111 | SELP | 1087G/A |
| RS5918 | 0.181 | 2.286 | 0.681 | 7.677 | 182 | 26.9231 | ITGB3 | 1565T/C |
| RS1799768 | 0.294 | 2.036 | 0.539 | 7.686 | 187 | 26.2032 | PAI-1 | −675indel(5G/4G) |
| RS1042714 | 0.587 | 1.705 | 0.509 | 5.713 | 185 | 25.4054 | ADRB2 | 79C/G |
| RS1800796 | 0.401 | 2.242 | 0.340 | 14.787 | 190 | 26.3158 | IL6 | −572G/C |
| RS1800947 | 0.427 | 0.405 | 0.044 | 3.763 | 189 | 26.4550 | CRP | 1059G/C |
| RS1205 | 0.452 | 0.623 | 0.182 | 2.137 | 165 | 26.6667 | CRP | 3′FI T/C |
| RS1800795 | 0.533 | 0.633 | 0.150 | 2.669 | 166 | 27.1084 | IL6 | −174G/C |
| RS3025058 | 0.548 | 0.675 | 0.187 | 2.433 | 145 | 28.2759 | MMP3 | −1171indel(5A/6A) |
| RS1800797 | 0.598 | 0.678 | 0.160 | 2.876 | 171 | 24.5614 | IL6 | −597G/A |
| RS6136 | 0.730 | 0.791 | 0.209 | 2.993 | 173 | 25.4335 | SELP | 2361A/C |
| RS1800629 | 0.846 | 1.128 | 0.336 | 3.786 | 182 | 25.8242 | TNFA | −308G/A |
| SNP030 | 0.931 | 0.947 | 0.275 | 3.257 | 178 | 26.4045 | ACE | (in16)indel(W/-) |
| RS2227631 | 0.935 | 0.952 | 0.293 | 3.090 | 187 | 26.7380 | PAI-1 | −844A/G |
| RS361525 | 0.984 | 1643515 | 0.000 | — | 175 | 26.8571 | TNFA | −238G/A |
| RS1042713 | 0.997 | 1.002 | 0.293 | 3.428 | 180 | 23.5556 | ADRB2 | 46A/G |

SNP—single nucleotide polymorphism
MACE—major adverse cardiac events
CABG—coronary artery bypass grafting

Thus, the risk of MACE after combined CABG and valvular surgery in women was significantly associated with a SNP in the P-selectin gene. The finding potentially identifies new mechanisms involved in the complex pathophysiology of cardiovascular disease and is expected to allow a more comprehensive risk assessment of cardiac surgical patients.

### Example 4

### Experimental Details

### Study Population

Patients enrolled in this study were part of the Perioperative Genetics and Safety Outcomes Study (PEGASUS), an ongoing Institutional Review Board approved, prospective, longitudinal study at Duke University Medical Center. The current substudy targeted a cohort of patients undergoing isolated coronary artery bypass graft (CABG) surgery utilizing cardiopulmonary bypass (CPB) during a specified period in whom detailed genotyping and cognitive testing was performed. Patients were excluded if they had a history of symptomatic cerebrovascular disease, psychiatric illness, renal failure, active liver disease, bleeding disorders, or had less than a seventh grade education.

### Patient Management

Anesthesia was induced and maintained with midazolam, fentanyl, and isoflurane. All patients underwent nonpulsatile hypothermic (30°-32° C.) CPB. The perfusion apparatus consisted of the Cobe CML membrane oxygenator™ (COBE Chem Labs, Lakewood, Colo.), the Sarns 7000 MDX pump™ (3M Inc, Ann Arbor, Mich.), and the Pall SP3840™ arterial line filter (Pall Biomedical Products Co, Glen Cove, N.Y.). Perfusion was maintained at pump flow rates of 2 to 2.4 L·min⁻¹·m² throughout CPB. The pump was primed with crystalloid, and serial hematocrits were kept at ≧0.18 with packed red blood cell transfusion as necessary. Arterial blood gases were measured every 15 to 30 minutes to maintain arterial carbon dioxide partial pressures of 35 to 40 mm Hg, unadjusted for temperature (α-stat), and oxygen partial pressures of 150 to 250 mm Hg.

### Measurement of Cognitive Function Phenotype

Cognitive function was assessed the day before surgery and at six weeks post-operatively by investigators experienced in neuropsychological testing, who were blinded to the genetic data. In accordance with the Consensus Statement on assessment of neurobehavioral outcomes after cardiac surgery (Murkin et al, Ann. Thorac. Surg. 59:1289-1295 (1995)), a cognitive test battery comprised of the following five instruments was used:
1) The Short Story module of the Randt Memory Test requires subjects to recall the details of a short story immediately after it has been read to them (immediate) and after a 30-minute delay (delay);
2) The Digit Span subtest of the Wechsler Adult Intelligence Scale-Revised (WAIS-R) Test requires subjects to repeat a series of digits that have been orally presented to them both forward and, in an independent test, in reverse order;
3) Modified Visual Reproduction Test from the Wechsler Memory Scale.

This test measures short- and long-term figural memory and requires subjects to reproduce from memory several geometric shapes both immediately and after a 30-minutes delay.
4) The Digit Symbol subtest of the WAIS-R is a paper and pencil task that requires subjects to reproduce, within 90 seconds, as many coded symbols as possible in blank boxes beneath randomly generated digits according to a coding scheme for pairing digits with symbols;
5) The Trail Making Test (Trails) (part B) requires subjects to connect, by drawing a line, a series of numbers, and letters in sequence (i.e., 1-A-2-B) as quickly as possible.

This cognitive test battery results in ten cognitive scores. To quantify changes in cognitive function over time while minimizing the potential for over-testing and redundancy in the cognitive measures, a factor analysis with orthogonal rotation was performed on the ten cognitive test scores from baseline, as previously described (Newman et al, N. Engl. J. Med. 344:395-402 (2001)). This factor analysis reduced the ten correlated scores to four uncorrelated domain scores. The factor loadings (weights) of each test at baseline were applied to the test scores from the 6-week evaluation to construct comparable 6-week domain scores. Cognitive deficit was defined as a decline of at least one standard deviation on any of the 4 domain scores at 6 weeks postoperatively.

### Candidate Polymorphism Selection

Based on review of the literature concerning cognitive function in experimental, clinical, and cardiac surgical settings, 37 polymorphisms were selected a priori as primary candidates for analysis of association with postoperative cognitive decline. These 37 polymorphisms, described in Table 10, represented 16 genes, which were classified on the basis of biological function into categories of inflammation, cell matrix adhesion/interaction, coagulation-thrombosis, lipid metabolism, and vascular reactivity.

TABLE 10
Polymorphisms and indels evaluated for their relationship to cognitive decline after CABG surgery.

| | | | | Minor Allele Frequency |
|---|---|---|---|---|
| Functional | | | Major/Minor | in White Patients Without |
| Category | SNP ID* | Gene Name | Allele | Cognitive Deficit |
| Inflammation | rs1205 | CRP (C-Reactive Protein) | 3′UTR 1846C/T | 0.338 |
| | rs1800947 | CRP (C-Reactive Protein) | 1059G/C | 0.080 |
| | rs1799942 | IL1A (Interleukin 1a) | 4844 G/T | 0.315 |
| | rs1800587 | IL1A (Interleukin 1a) | −889 C/T | 0.317 |
| | rs2229234 | IL1RN (Interleukin 1 Receptor Antagonist) | 11100 T/C | 0.302 |
| | rs2229235 | IL1RN (Interleukin 1 Receptor Antagonist) | 8006T/C | 0.297 |
| | rs1800795 | IL6 (Interleukin 6) | −174G/C | 0.473 |
| | rs1800796 | IL6 (Interleukin 6) | −572G/C | 0.044 |
| | rs1800797 | IL6 (Interleukin 6) | −597G/A | 0.469 |
| | rs1800610 | TNFA (Tumor Necrosis Factor alpha) | +488G/A | 0.075 |
| | rs1800629 | TNFA (Tumor Necrosis Factor alpha) | −308G/A | 0.153 |
| | rs1800750 | TNFA (Tumor Necrosis Factor alpha) | −376G/A | 0.009 |
| | rs361525 | TNFA (Tumor Necrosis Factor alpha) | −238G/A | 0.070 |
| Cell Matrix | rs3025058 | MMP3 (Matrix Metalloproteinase 3) | −1171indel (5A/6A) | 0.500 |
| Adhesion/ | SNP028** | MMP9 (Matrix Metalloproteinase 9) | −1562C/T | 0.119 |
| Interaction | rs1800805 | SELP (P-selectin) | −1969A/G | 0.412 |
| | rs6127 | SELP (P-selectin) | 1902A/G | 0.391 |
| | rs6131 | SELP (P-selectin) | 1087G/A | 0.183 |
| | rs6133 | SELP (P-selectin) | 2013G/T | 0.117 |
| | rs6136 | SELP (P-selectin) | 2361A/C | 0.117 |
| Coagulation- | rs2243093 | GP1BA (Glycoprotein Ib alpha) | −5 T/C | 0.102 |
| Thrombosis | rs6065 | GP1BA (Glycoprotein Ib alpha) | 524C/T | 0.075 |
| | rs1613662 | GP6 (Glycoprotein VI) | 13254T/C | 0.186 |
| | rs1800198 | ITGA2 (Glycoprotein IaIIa) | 807C/T | 0.357 |
| | rs28095 | ITGA2 (Glycoprotein IaIIa) | −52C/T | 0.346 |
| | rs5918 | ITGB3 (Glycoprotein IIIa) | 1565T/C | 0.167 |
| | rs1799768 | PAI1 (Plasminogen Activator Inhibitor 1) | −675indel (5G/4G) | 0.459 |
| | rs2227631 | PAI1 (Plasminogen Activator Inhibitor 1) | −844A/G | 0.428 |
| Lipid | rs405509 | APOE (Apolipoprotein E) | −219G/T | 0.475 |
| Metabolism | rs429358 | APOE (Apolipoprotein E) | 448T/C | 0.125 |
| | rs7412 | APOE (Apolipoprotein E) | 586C/T | 0.071 |
| Vascular | rs4291 | ACE (Angiotensin Converting Enzyme) | −240A/T | 0.405 |
| Reactivity | rs4344 | ACE (Angiotensin Converting Enzyme) | 2350G/A | 0.421 |
| | SNP030** | ACE (Angiotensin Converting Enzyme) | (in16) indel (D/I) | 0.416 |
| | rs1799983 | NOS3 (Endothelial Nitric Oxide Synthase) | 894G/T | 0.318 |
| | rs1799985 | NOS3 (Endothelial Nitric Oxide Synthase) | 10(in23)G/T | 0.307 |
| | rs2070744 | NOS3 (Endothelial Nitric Oxide Synthase) | −786T/C | 0.356 |

*From NCBI's dbSNP public database (ncbi.nlm.nih.gov/SNP/)
**Duke polymorphism ID number

### Isolation of Genomic DNA and Genotype Analysis

Whole blood was collected preoperatively with genomic DNA extraction subsequently performed using the Puregene™ system (Gentra Systems, Minneapolis, Minn.). Genotyping assays were conducted at Agencourt Bioscience Corporation (Beverly, Mass.) by Matrix Assisted Laser Desorption/Ionization Time-Of-Flight (MALDI-TOF) mass spectrometry, using the Sequenom MassArray™ system (Sequenom, San Diego, Calif.) (Sun et al, Nucleic Acids Res. 28:E68 (2000)). Primers used and polymorphism details can be found in Table 11.

TABLE 11
Genotyping assays for the candidate gene polymorphisms investigated

| | | Major/Minor | Genotyping Assay |
|---|---|---|---|
| Gene Name | SNP ID¹ | Allele | Primers² |
| CRP (C-Reactive Protein) | rs1205 | 3′UTR 1846C/T | ACGTTGGATGGCCATCTTGTTTGCCACATG |
| | | | ACGTTGGATGGTTTGTCAATCCCTTGGCTC |
| | | | TTGTTTGCCACATGGAGAGAGACT |
| CRP (C-Reactive Protein) | rs1800947 | 1059G/C | ACGTTGGATGGAAATGTGAACATGTGGGAC |
| | | | ACGTTGGATGAGGACATTAGGACTGAAGGG |
| | | | TGTGAACATGTGGGACTTTGTGCT |
| IL1A (Interleukin 1a) | rs1799942 | 4844 G/T | ACGTTGGATGTTTCACATTGCTCAGGAAGC |
| | | | ACGTTGGATGATCTGCACTTGTGATCATGG |
| | | | GCTCAGGAAGCTAAAAGGTG |
| IL1A (Interleukin 1a) | rs1800587 | −889 C/T | ACGTTGGATGTTGGGAGAAAGGAAGGCATG |
| | | | ACGTTGGATGTTCTACCACCTGAACTAGGC |
| | | | TTTTTACATATGAGCCTTCAATG |
| IL1RN (Interleukin 1 | rs2229234 | 11100 T/C | ACGTTGGATGGAACAGAAAGCAGGACAAGC |
| Receptor Antagonist) | | | ACGTTGGATGAGGCGGCAGACTCAAAACTG |
| | | | CGCCTTCATCCGCTCAGACAG |
| IL1RN (Interleukin 1 | rs2229235 | 8006T/C | ACGTTGGATGTGGGATGTTAACCAGAAGAC |
| Receptor Antagonist) | | | ACGTTGGATGAATTGACATTTGGTCCTTGC |
| | | | CTGAGGAACAACCAACTAGTTGC |
| IL6 (Interleukin 6) | rs1800795 | −174G/C | ACGTTGGATGAGCCTCAATGACGACCTAAG |
| | | | ACGTTGGATGGATTGTGCAATGTGACGTCC |
| | | | TTTCCCCCTAGTTGTGTCTTGC |
| IL6 (Interleukin 6) | rs1800796 | −572G/C | ACGTTGGATGACGCCTTGAAGTAACTGCAC |
| | | | ACGTTGGATGTCTTCTGTGTTCTGGCTCTC |
| | | | CAGGCAGTCTACAACAGCC |
| IL6 (Interleukin 6) | rs1800797 | −597G/A | ACGTTGGATGACGCCTTGAAGTAACTGCAC |
| | | | ACGTTGGATGTCTTCTGTGTTCTGGCTCTC |
| | | | AAGTAACTGCACGAAATTTGAGG |
| TNFA (Tumor Necrosis | rs1800610 | +488G/A | ACGTTGGATGGAAAGATGTGCGCTGATAGG |
| Factor alpha) | | | ACGTTGGATGCTTGCCACATCTCTTTCTGC |
| | | | GGGAGGGATGGAGAGAAAAAAAC |
| TNFA (Tumor Necrosis | rs1800629 | −308G/A | ACGTTGGATGGATTTGTGTGTAGGACCCTG |
| Factor alpha) | | | ACGTTGGATGGGTCCCCAAAAGAAATGGAG |
| | | | ACCCTGGAGGCTGAACCCCGTCC |
| TNFA (Tumor Necrosis | rs1800750 | −376G/A | ACGTTGGATGCTCCCAGTTCTAGTTCTATC |
| Factor alpha) | | | ACGTTGGATGTTGCCTCCATTTCTTTTGGG |
| | | | TTCCTGCATCCTGTCTGGAA |
| TNFA (Tumor Necrosis | rs361525 | −238G/A | ACGTTGGATGACACAAATCAGTCAGTGGCC |
| Factor alpha) | | | ACGTTGGATGATCAAGGATACCCCTCACAC |
| | | | GAAGACCCCCCTCGGAATC |
| MMP3 (Matrix | rs3025058 | −117lindel | ACGTTGGATGGTCCTCATATCAATGTGGCC |
| Metalloproteinase 3) | | (5A/6A) | ACGTTGGATGCTATGGTTCTCCATTCCTTTG |
| | | | GGACAAGACATGGTTTTT |
| MMP9 (Matrix | SNP028³ | −1562C/T | ACGTTGGATGAAAAATTTAGCCAGGCGTGG |
| Metalloproteinase 9) | | | ACGTTGGATGGGTTCAAGCAATTCTCCTGC |
| | | | CCAGGCGTGGTGGCGCA |
| SELP (P-selectin) | rs1800805 | −1969A/G | ACGTTGGATGGCCTCCATGTCTTTCTGTTC |
| | | | ACGTTGGATGTCTTCATCCCCATCTGAGAC |
| | | | GTTCTTACTTTCACTCACCAGC |
| SELP (P-selectin) | rs6127 | 1902A1G | ACGTTGGATGTTCAATGTTGGCTCCACCTG |
| | | | ACGTTGGATGGCATTCCACATTATTGGGCC |
| | | | GCTCCACCTGTCATTTCTCTTGT |
| SELP (P-selectin) | rs6131 | 1087G/A | ACGTTGGATGGCAAAAGCAGTGAGCGGATG |
| | | | ACGTTGGATGTCTCCAGCTGTGCAGTGTCAG |
| | | | TGAACACAGTCCATGGTTCCTTCA |
| SELP (P-selectin) | rs6133 | 2013G/T | ACGTTGGATGTACAGTACATGGTTCCCTGC |
| | | | ACGTTGGATGACAGGCATAGCATCACTTCC |
| | | | GGTGAGGGCTGGACATTGCA |
| SELP (P-selectin) | rs6136 | 2361A/C | ACGTTGGATGATGCCAAGAGAATGGCCAC |
| | | | ACGTTGGATGCCTGCTTGGCAGGTTGGCA |
| | | | AAGAGAATGGCCACTGGTCA |
| GP1BA (Glycoprotein Ib | rs2243093 | −5 T/C | ACGTTGGATGATCCACTCAAGGCTCCCTTG |
| alpha) | | | ACGTTGGATGTTGGCAGCAGGAGCAGCAAG |
| | | | GGCTCCCTTGCCCACAGG |
| GP1BA (Glycoprotein Ib | rs6065 | 524C/T | ACGTTGGATGTGTTGTTAGCCAGACTGAGC |
| alpha) | | | ACGTTGGATGAAGGCAATGAGCTGAAGACC |
| | | | TCCAGCTTGGGTGTGGGC |
| GP6 (Glycoprotein VI) | rs1613662 | 13254T/C | ACGTTGGATGATTTCCCAGGAACCTCTGTG |
| | | | ACGTTGGATGATACGCTGTGCACCAGAATG |
| | | | TACCAACAGAACCACCTTCC |
| ITGA2 (Glycoprotein IaIIa) | rs1800198 | 807C/T | ACGTTGGATGTGGCCTATTAGCACCAAAAC |
| | | | ACGTTGGATGAGACATCCCAATATGGTGGG |
| | | | TTACCTTGCATATTGAATTGCTCC |
| ITGA2 (Glycoprotein IaIIa) | rs28095 | −52C/T | ACGTTGGATGGATCCGGTGTTTGCGGAATC |
| | | | ACGTTGGATGAGGGAAAAGTTTCTGGGCAG |
| | | | CGGAATCAGGAGGGGCGGGC |
| ITGB3 (Glycoprotein IIIa) | rs5918 | 1565T/C | ACGTTGGATGCCTTCAGCAGATTCTCCTTC |
| | | | ACGTTGGATGTTGCTGGACTTCTCTTTGGG |
| | | | TCACAGCGAGGTGAGCCC |
| PAI1 (Plasminogen | rs1799768 | −675 indel | ACGTTGGATGCTCCGATGATACACGGCTGA |
| Activator Inhibitor 1) | | (5G/4G) | ACGTTGGATGAGGTTGTTGACACAAGAGAG |
| | | | GATACACGGCTGACTCCCC |
| PAI1 (Plasminogen | rs2227631 | −844A/G | ACGTTGGATGAAGGAAACAGGAGACCAACG |
| Activator Inhibitor 1) | | | ACGTTGGATGGAGGATAAAGGACAAGCTGC |
| | | | GACCAACGTGTAAGTTTCACTTC |
| APOE (Apolipoprotein E) | rs405509 | −219G/T | ACGTTGGATGACATTCCCCTTCCACGCTTG |
| | | | ACGTTGGATGTAGAGGTCTTTTGACCACCC |
| | | | GAATGGAGGAGGGTGTCTG |
| APOE (Apolipoprotein E) | rs429358 | 448T/C | ACGTTGGATGTGTCCAAGGAGCTGCAGGC |
| | | | ACGTTGGATGTCGGTGCTCTGGCCGAGCAT |
| | | | GCGGACATGGAGGACGTG |
| APOE (Apolipoprotein E) | rs7412 | 586C/T | ACGTTGGATGACATTCCCCTTCCACGCTTG |
| | | | ACGTTGGATGTAGAGGTCTTTTGACCACCC |
| | | | GAATGGAGGAGGGTGTCTG |
| ACE (Angiotensin | rs4291 | −240A/T | ACGTTGGATGGCAGAGGAAGCTGGAGAAAG |
| Converting Enzyme) | | | ACGTTGGATGTCGGGTGTTCCGGCAAACTG |
| | | | CTGGAGAAAGGGCCTCCTCTCTTT |
| ACE (Angiotensin | rs4344 | 2350G/A | ACGTTGGATGACAATGTTGTGATGGGTGCC |
| Converting Enzyme) | | | ACGTTGGATGATCTATGTCGGGCAAGTCAC |
| | | | CAAGATTATTAACTTCTTCCCC |
| ACE (Angiotensin | SNP030³ | (in16) indel | CTGGAGACCACTCCCATCCTTTCT |
| Converting Enzyme) | | (D/I)⁴ | GATGTGGCCATCACATTCGTCAGAT |
| NOS3 (Endothelial Nitric | rs1799983 | 894G/T | ACGTTGGATGAAACGGTCGCTTCGACGTGC |
| Oxide Synthase) | | | ACGTTGGATGATCCCTTTGGTGCTCACGTG |
| | | | GCTGCAGGCCCCAGATGA |
| NOS3 (Endothelial Nitric | rs1799985 | 10(in23)G/T | ACGTTGGATGAGCGGCTGCATGACATTGAG |
| Oxide Synthase) | | | ACGTTGGATGGTCCCTAGATTGTGTGACTC |
| | | | TGAGAGCAAAGGTGAGGCTG |
| NOS3 (Endothelial Nitric | Rs207074 | −786T/C | ACGTTGGATGAGTTTCCCTAGTCCCCCATG |
| Oxide Synthase) | | | ACGTTGGATGAGTCAGCACAGAGACTAGGG |
| | | | CATCAAGCTCTTCCCTGGC |

¹From NCBI's dbSNP public database (http://www.ncbi.nlm.nih.gov/SNP/)
²MALDI-TOF mass spectrometry genotyping assays: from top to bottom - forward PCR primer, reverse PCR primer, extension primer.
³Duke internal polymorphism ID number.
⁴NOTE: The ACE indel polymorphism was genotyped using PCR amplification, size fractionation and electrophoretic visualization as described.
PCR-polymerase chain reaction

Genotype accuracy of the Sequenom MassArray™ system was estimated at 99.6% (Gabriel et al, Science 296:2225-2229 (2002)). Using direct sequencing on an AB13700 capillary sequencer (Applied Biosystems, Foster City, Calif.), genotyping reproducibility in this study was validated to be >99% by scoring a panel of 6 polymorphisms in 100 randomly selected patients. The ACE insertion/deletion polymorphism was genotyped by polymerase chain reaction amplification of the respective fragments from intron 16 of the ACE gene, followed by size-fractionation through electrophoresis, as previously described (Rigat et al, Nucleic Acids Res. 20:1433 (1992)). Results were scored by two independent investigators blinded to clinical phenotype.

### Statistical Analysis

Categorical and continuous demographic characteristics were compared between deficit groups with Pearson Ch-Square and Wicoxon rank sum tests, respectively. To test for association between the 37 candidate gene polymorphisms and incidence of postoperative cognitive decline, a two-stage analysis approach was employed: marker selection, followed by model building (Hoh et al, Ann. Hum. Genet. 64:413-417 (2000)).

First, descriptive statistics, including allele and genotype frequencies were calculated for each polymorphism, and Hardy-Weinberg equilibrium was evaluated using an exact test among unaffected patients (Guo and Thompson, Biometrics 48:361-372 (1992)). All analyses were based on two genotypic classes, distinguished by the presence or absence of at least one copy of the least frequent or minor allele (homozygote minor and heterozygote versus homozygote major). The genotype frequencies between patients with and without cognitive deficit were then compared using univariate Pearson's exact Chi-Squared test for each of the 37 polymorphisms, and selected a set of markers with p-value<0.2.

Second, a series of logistic regression analyses were performed to test the independent association of all pairs of markers selected in the previous step with occurrence of cognitive deficit. Because the analysis strategy employed many separate tests of independence, an adjustment of observed p-values was required to account for this multiple testing. Random permutation analysis was used to adjust p-values from the logistic regressions. Four thousand copies of the data set were generated, randomly reassigning cognitive deficit to study subjects, thereby dissociating genotype from phenotype in the copies. For each permutation, p-values were calculated from logistic models with each pair of SNPs. For each SNP pair, the smallest model p-value was retained to estimate the distribution of “smallest” 2 SNP model p-values under the null hypothesis of no association. An adjusted p-value was computed as the fraction of permutation p-values that were smaller than the observed p-value. For example, if among 4000 permutations, 40 p-values were smaller than the observed p-value, then the adjusted p-value would be 40/4000 or 0.01.

To account for population substructure, which can result in false positive associations, self-reported ethnicity was included as a covariate in multiple logistic regression models. Patients representing ethnic groups other than White, African American, and Native American were excluded from analyses involving race. Furthermore, the structured association method was employed to control for the possibility of cryptic heterogeneity within the White patients (Pritchard et al, Am. J. Hum. Genet. 67:170-181 (2000)); multilocus genotypes from a panel of 52 unlinked biallelic null markers, evenly distributed across the genome, were used to identify clusters of genetically similar individuals. Posterior probabilities of subpopulation membership (based on 2 putative populations) were subsequently used in the logistic regression models that included age, baseline cognitive score, years of education, and race as covariates.

All statistical analysis was performed using SAS and SAS/Genetics version 8.02 (SAS Inc, Cary, N.C.). Population structure was investigated using the Structure program (version 2.1), available at pritch.bsd.uchicago.edu/software.html. Continuous variables were described as mean±standard deviation; categorical variables were described as percentages.

### Post-Hoc Analyses

In a subset of 93 patients, platelet activation was measured in serial arterial blood samples that had been collected at the following time points: prior to induction of anesthesia, before aortic cross-clamp release, 10 minutes after cross-clamp release, at the end of CPB, and at the end of surgery. After incubation with saturating concentrations of monoclonal antibodies, blood samples were analyzed on a FACScan flow cytometer. The percentage of platelets expressing P-selectin (CD62P) was determined as a marker of platelet activation. Similarly, CRP levels were measured in 336 patients from whom serum had been collected prior to induction of anesthesia and at 4.5, 24 and 48 hours after cross-clamp removal. Collected blood was centrifuged and the resultant supernatant was immediately frozen at −70° C. until analysis was completed. Immunoassays were forward immunometric (sandwich) assays performed by Biosite Diagnostics (San Diego, Calif.) in a 10-μl reaction volume in 384-well microtiter plates using a Tecan Genesis RSP 200/8 Workstation (Tecan, Research Triangle Park, N.C.).

The association of percentage of P-selectin-positive platelets with SELP (P-selectin) genotype and of serum CRP levels with CRP genotype was tested using repeated measures analysis of variance (ANOVA) based on log transformation of the skewed data and using an unstructured covariance model. Because baseline concentrations differed between SELP genotypes, concentration levels at subsequent times were expressed as ratio to baseline. Genotype differences at individual event times were tested using Wilcoxon rank sum tests when ANOVA was significant at p<0.05.

### Results

Demographic characteristics of the 680 enrolled patients are presented in Table 12. Of these, 164 (24%) did not complete 6-week testing, leaving 516 patients for the final analyses; as in prior studies non-returners were older and sicker. The incidence of cognitive deficit in the study sample was 35.7% (184/516) and patients with a deficit were similar to those without a deficit, with the exception of baseline cognition (Table 12).

TABLE 12
Demographic characteristics of the study population (values are expressed as mean (SD) or as (%).

| | | | | | | | | | CogDef | Return |
|---|---|---|---|---|---|---|---|---|---|---|
| | All Patients: | | Cognitive Deficit | | | | No 6 Wk Test^{#} | | Group | Group |
| | N = 680 | | No: N = 332 | | Yes: N = 184 | | N = 164 | | P-value* | P-value** |
| Age (yrs) | 61.7 | (10.4) | 60.7 | (10.3) | 61.1 | (10.5) | 64.2 | (10.3) | 0.7793 | 0.0003 |
| Height (cm) | 171.6 | (10.2) | 171.7 | (10.5) | 172.3 | (9.5) | 170.5 | (10.4) | 0.7167 | 0.1548 |
| Weight (kg) | 86.3 | (18) | 86.1 | (18.0) | 87.9 | (17.8) | 85.0 | (18.0) | 0.4337 | 0.1837 |
| Ejection Fraction (%) | 54.3 | (12.4) | 54.4 | (11.9) | 55.4 | (12.1) | 52.4 | (13.7) | 0.4347 | 0.0999 |
| Bypass Time (mins) | 109.4 | (37.2) | 107.6 | (34.2) | 104.6 | (30.5) | 118.5 | (47.2) | 0.5016 | 0.0027 |
| Cross-Clamp Time (mins) | 58.0 | (23.8) | 56.8 | (23.6) | 54.8 | (20.5) | 64.2 | (26.3) | 0.4512 | 0.0004 |
| # Grafts | 3.2 | (0.9) | 3.1 | (0.9) | 3.1 | (0.9) | 3.3 | (0.8) | 0.6549 | 0.1483 |
| Years of Education | 12.4 | (3.2) | 12.4 | (3.2) | 12.8 | (3.1) | 11.8 | (3.2) | 0.2008 | 0.0062 |
| Baseline Cognition | −0.002 | (0.515) | −0.008 | (0.487) | 0.179 | (0.456) | −0.193 | (0.562) | <0.0001 | <0.0001 |

| | | | | | | |
|---|---|---|---|---|---|---|
| Female Gender (%) | 30.3 | 30.4 | 26.6 | 34.1 | 0.4182 | 0.2418 |
| Prior CABG (%) | 3.2 | 3.6 | 2.2 | 3.7 | 0.4370 | 0.7850 |
| Prior MI (%) | 29.2 | 28.1 | 21.8 | 40.1 | 0.1544 | 0.0012 |
| Prior Stroke/TIA (%) | 2.1 | 1.3 | 1.1 | 4.7 | 0.9999 | 0.0164 |
| Unstable Angina (%) | 60.4 | 57.6 | 66.1 | 59.7 | 0.0666 | 0.8487 |
| Diabetes (%) | 30.1 | 26.4 | 29.0 | 39.0 | 0.5361 | 0.0056 |
| CHF (%) | 11.8 | 10.3 | 13.8 | 12.8 | 0.2996 | 0.7718 |
| Hypertension (%) | 62.5 | 61.4 | 61.5 | 65.8 | 0.9999 | 0.3842 |
| COPD (%) | 9.3 | 9.3 | 4.6 | 14.8 | 0.0739 | 0.0106 |
| PVD (%) | 12.3 | 12.2 | 8.0 | 17.4 | 0.1708 | 0.0328 |
| Race: | | | | | 0.1517 | 0.4036 |
| White (%) | 86.0 | 88.3 | 83.2 | 84.8 | | |
| African American (%) | 9.7 | 8.4 | 9.8 | 12.2 | | |
| Native American (%) | 3.7 | 3.0 | 5.4 | 3.1 | | |
| Other (%) | 0.6 | 0.3 | 1.6 | 0 | | |

MI = myocardial infarction; TIA = transient ischemic attack; CHF = congestive heart failure; COPD = chronic obstructive pulmonary disease; PVD = peripheral vascular disease
^{#}Non-return group: deceased (13), incomplete testing (3), poor health (31), unable to travel (17), unwilling to return (43), unable to contact (38), other (19)
*CogDef Group P-values compares patients with and without cognitive deficit
**Return Group P-values compares patients with and without 6-week cognitive tests

Initial demographic comparisons revealed differences in cognitive deficit among racial groups (White—34.3%, African American—39.1%, Native American—50%) after accounting for age, baseline cognition, and years of education in multivariable logistic regression. Therefore, all subsequent analyses were limited to the subset of White patients (N=447). Minor allele frequencies for the 37 polymorphisms examined among the White patients without cognitive deficit are presented in Table 10. Four polymorphisms had deviations from Hardy-Weinberg equilibrium and were excluded from subsequent analyses. Based on the first step of the selection process, 13 markers were selected for modeling, and are presented in Table 13.

TABLE 13
List of the 13 markers chosen for final modeling
and their corresponding genotype frequencies.

| | | FREQUENCY IN WHITES | |
|---|---|---|---|
| | | With | Without |
| SNP ID | GENOTYPE | Cognitive Deficit | Cognitive Deficit |
| Rs1799768 | 4G/4G | 0.230 | 0.275 |
| PAI1 | 4G/5G | 0.453 | 0.532 |
| −675indel | 5G/5G | 0.318 | 0.193 |
| rs2227631 | A/A | 0.212 | 0.278 |
| PAI1 | A/G | 0.582 | 0.588 |
| −844A/G | G/G | 0.205 | 0.134 |
| rs1800198 | C/C | 0.432 | 0.404 |
| ITGA2 | C/T | 0.379 | 0.478 |
| 807C/T | T/T | 0.189 | 0.118 |
| rs1800610 | A/A | 0.014 | 0.000 |
| TNFA | A/G | 0.130 | 0.150 |
| 488G/A | G/G | 0.855 | 0.850 |
| rs361525 | A/A | 0.000 | 0.004 |
| TNFA | A/G | 0.064 | 0.132 |
| −238G/A | G/G | 0.936 | 0.864 |
| rs1800797 | A/A | 0.127 | 0.188 |
| IL6 | A/G | 0.530 | 0.563 |
| −597G/A | G/G | 0.343 | 0.249 |
| rs1800947 | C/C | 0.007 | 0.004 |
| CRP | C/G | 0.075 | 0.153 |
| 1059G/C | G/G | 0.918 | 0.844 |
| rs2229235 | C/C | 0.070 | 0.081 |
| IL1RN | C/T | 0.371 | 0.432 |
| 8006T/C | T/T | 0.559 | 0.487 |
| rs4291 | A/A | 0.321 | 0.278 |
| ACE | A/T | 0.641 | 0.633 |
| −240A/T | T/T | 0.038 | 0.089 |
| rs6127 | A/A | 0.331 | 0.356 |
| SELP | A/G | 0.591 | 0.506 |
| 1902A/G | G/G | 0.079 | 0.138 |
| rs6131 | A/A | 0.035 | 0.034 |
| SELP | A/G | 0.167 | 0.298 |
| 1087G/A | G/G | 0.799 | 0.668 |
| rs405509 | G/G | 0.389 | 0.318 |
| APOE | G/T | 0.349 | 0.415 |
| −219G/T | T/T | 0.262 | 0.267 |
| rs7412 | C/C | 0.828 | 0.861 |
| APOE | C/T | 0.152 | 0.135 |
| 586C/T | T/T | 0.021 | 0.004 |

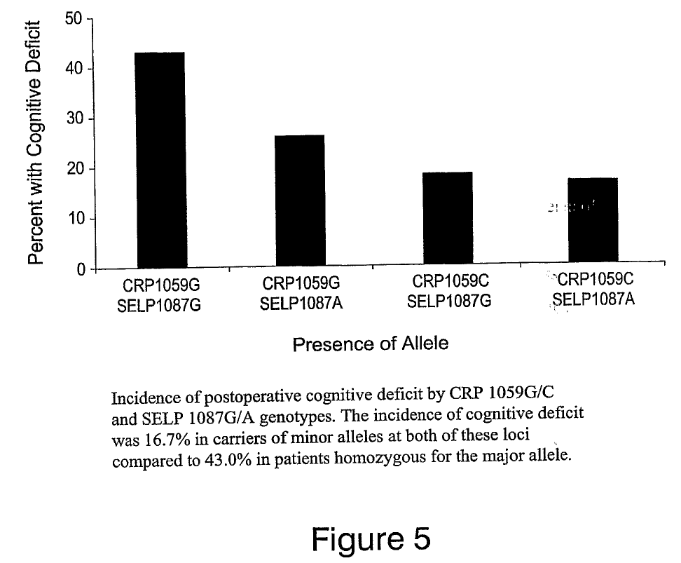

Logistic regression in Whites alone revealed that in addition to age, baseline cognition, and years of education, a CRP 1059G/C SNP and a SELP 1087G/A SNP were significantly associated with cognitive deficit (area under the ROC curve=0.70; p<0.0001; Table 14). Presence of the minor allele at both CRP and SELP polymorphisms had a protective effect; the incidence of cognitive deficit was 16.7% in carriers of minor alleles at both of these loci compared to 43.0% in patients homozygous for the major allele (FIG. 5). Thus, the absolute risk reduction in experiencing postoperative cognitive deficit was 20.5% in carriers of the CRP 1059C allele and 15.4% for the SELP 1087A allele. In the final logistic regression model, the interaction term between this pair of SNPs was not significant. After adjustment for multiple comparisons using permutation testing, the independent association of this pair of CRP and SELP genotypes to cognitive deficit remained significant (adjusted p=0.030).

TABLE 14
Predictors of cognitive deficit after cardiac surgery.

| Variable | Odds Ratio | 95% Confidence Interval | P-value |
|---|---|---|---|
| CRP 1059G/C SNP | 0.37 | 0.16-0.78 | 0.013 |
| SELP 1087G/A SNP | 0.50 | 0.30-0.83 | 0.008 |
| Years of Education | 0.91 | 0.83-0.99 | 0.024 |
| Age (per year) | 1.03 | 1.01-1.05 | 0.017 |
| Baseline Cognition | 5.57 | 2.89-10.88 | <0.001 |

In separate logistic regression models which included self-reported race or subpopulation membership probabilities in Whites, the SNP effects remained significant. However, none of the two-way interactions between CRP and SELP SNPs with race or subpopulation membership was significant, suggesting that race and population substructure had no effect on the SNP associations described.

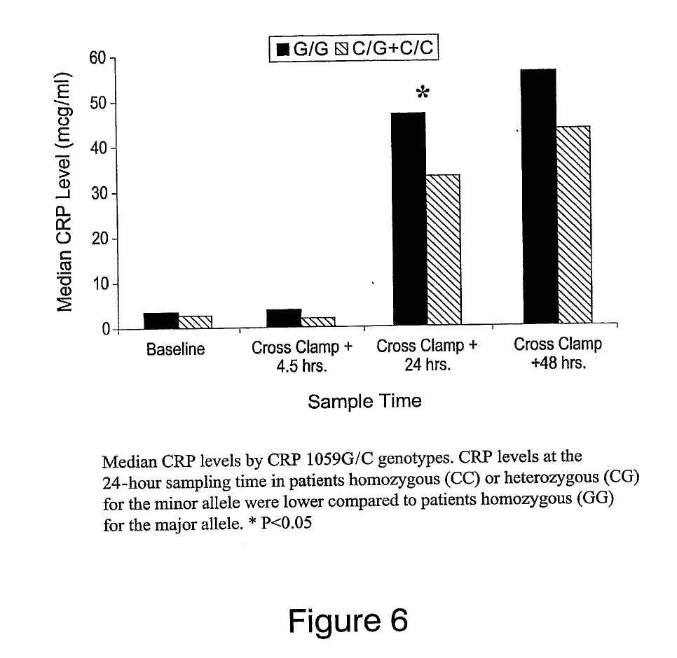

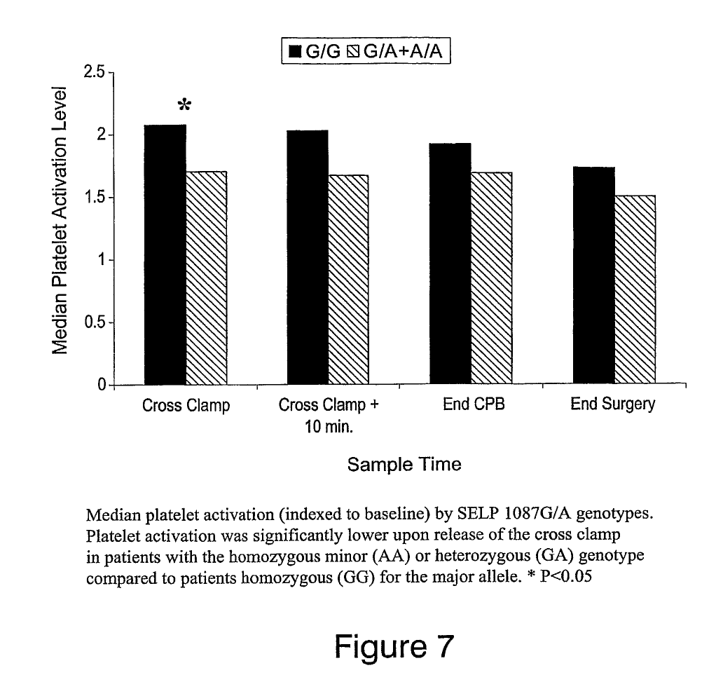

For serum CRP levels, a significant interaction between time and CRP 1059G/C genotype was seen in the repeated measures ANOVA (p=0.0106). The post-hoc Wilcoxon tests showed significantly lower serum CRP levels at the 24-hour sampling time in patients homozygous (CC) or heterozygous (CG) for the minor allele compared to patients homozygous (GG) for the major allele (p=0.0005, FIG. 6). Similarly, a significant genotype and time difference was seen when the effect of the SELP 1087G/A genotype on the percentage of P-selectin-positive platelets was examined. Platelet activation was significantly lower upon release of the cross clamp (p=0.0432) in patients with the homozygous minor (AA) or heterozygous (GA) genotype compared to patients homozygous (GG) for the major allele. (See FIG. 7.)

Summarizing, cognitive dysfunction remains a common complication following cardiac surgery, occurring in approximately 36% of patients at 6 weeks after surgery. In the study described above, of 516 CABG patients tested pre- and postoperatively with a detailed cognitive test battery, a genetic basis for this cognitive decline is reported. In addition to the previously described risk factors of age, level of education and baseline cognition it was found that the risk of cognitive deficit was significantly lower in patients carrying at least one copy of the CRP 1059C or SELP 1087A alleles. Furthermore, it was demonstrated that the corresponding intermediate phenotypes, perioperative serum levels of CRP and platelet activation, were reduced in patients with these polymorphisms, providing plausibility to the observed allelic association. This confluence of genetic and environmental risk factors suggests an integrated etiological model consistent with the concept of cognitive reserve (Stern, J. Int. Neuropsychol Soc. 8:448-460 (2002)). In the presence of favorable environments (such as higher educational and occupational attainment) and genetic makeup, individuals develop cognitive reserve, which increases their threshold for adverse neuropsychological responses to brain injury. Supported by epidemiologic evidence (Stern et al, JAMA 271:1004-1010 (1994)), structural and functional imaging studies (Scarmeas et al, Neuroimage 19:1215-1227 (2003), Stern et al, Cereb. Cortex 15:394-402 (2005)), as well as familial aggregation and twin studies (Lee, J. Clin. Exp. Neuropsychol. 25:594-613 (2003), cognitive reserve is thus a complex trait which may explain the observed variability in response to many forms of neurological insult, including cardiac surgery.

CABG surgery employing CPB is associated with an ischemia-reperfusion injury, inducing a complex inflammatory response that impacts not only the heart but also the brain, lungs, kidneys, and gut (Herskowitz and Mangano, Anesthesiology 85:957-960 (1996)). CRP is an acute-phase reactant produced primarily in the liver in response to tissue injury or inflammation, recently implicated not only as a marker, but also a potential participant in the pathogenesis of inflammatory-mediated processes (Pepys and Hirschfield, J. Clin. Invest. 111:1805-1812 (2003)). Mean CRP concentrations have been reported to rise as much as 83-fold from the preoperative period to 72 hours after CABG surgery and this variation appears to be influenced by CRP genotype (Brull et al, Arterioscler. Thromb. Vasc. Biol. 23:2063-2069 (2003)). The human CRP gene is located on chromosome 1q23 and consists of two exons and one intron (Cao and Hegele, J. Hum. Genet. 45:100-101 (2000)). Many CRP polymorphisms have been reported in the literature but none are thought to encode for an amino acid change. The 1059G/C SNP in exon 2 results in a codon change from CTG to CTC, both of which encode for leucine (Suk et al, Atherosclerosis 178:139-145 (2005)). This SNP, however, has been associated with lower CRP levels. Zee and Ridker (Zee and Ridker, Atherosclerosis 162:217-219 (2002)), using 726 case control pairs of initially healthy American males reported that median CRP levels were significantly lower (1.05 vs 1.38 mg/L) among heterozygotes (GC) than among homozygotes (GG). Similarly, from a study of 2397 participants, Suk et al (Atherosclerosis 178:139-145 (2005)) reported a 29% lower baseline CRP level in the presence of the less frequent C allele.

Immune activation has been demonstrated in experimental conditions to produce alterations in emotional states and decreased performance on memory tests. In a double-blind crossover study of 20 healthy male volunteers given an intravenous injection of endotoxin, significant positive correlations were found between cytokine secretion and endotoxin-induced decreased verbal and nonverbal memory functions (Reichenberg et al, Arch. Gen. Psychiatry 58:445-452 (2001)). These findings are consistent with reports that memory impairment is a common adverse effect of cytokine therapy and viral infection (Capuron et al, Psychol. Med. 29:291-297 (1999), Dantzer et al, Cytokines, stress, and depression, x,”, p. 336 (Kluwer Academic/Plenum Publishers, New York, (1999)). Inflammatory mechanisms and immune activation have been hypothesized to play a role not only in pathological conditions such as Alzheimer's Disease (Singh and Guthikonda, J. Psychiatr. Res. 31:657-660 (1997)), and vascular dementia (Schmidt et al, Ann. Neurol. 52:168-174 (2002)) but also in normal aging (Teunissen et al, J. Neuroimmunol. 134:142-150 (2003), Tilvis et al, J. Gerontol. A Biol. Sci. Med. Sci. 59:268-274 (2004), Yaffe et al, Neurology 61:76-80 (2003)). Yaffe et al (Neurology 61:76-80 (2003)) followed 3031 well-functioning subjects for 2 years and reported that a high baseline level of serum markers of inflammation, most notably CRP, was associated with poor cognitive performance and greater risk of cognitive decline over the follow-up period. Similar associations between elevated baseline CRP levels and greater cognitive decline have been reported in patients followed for 5-6 years (Teunissen et al, J. Neuroimmunol. 134:142-150 (2003), Tilvis et al, J. Gerontol. A Biol. Sci. Med. Sci. 59:268-274 (2004)). In the above-described study, the lower incidence of cognitive deficit in patients with the CRP 1059G/C polymorphism may be related to a lesser degree of inflammation as evidenced by the lower levels of serum CRP.

Selectins are a family of three different glycoproteins (E-, L-, and P-selectin) sharing a conserved structure (Blankenberg et al, Atherosclerosis 170:191-203 (2003)). The largest of these is P-selectin (CD62P), a component of the membrane of alpha and dense granules of platelets, which is also found in the membrane of the Weibel-Palade bodies of endothelial cells. Numerous experiments in knockout mice and humans have clearly illustrated the role of P-selectin in supporting platelet-leukocyte interactions and in leukocyte rolling on the endothelium (Blann et al, Eur. Heart J. 24:2166-2179 (2003)). P-selectin expression on activated platelets appears also to be important for the formation of large and stable platelet aggregates, for the amplification of the leukocyte recruitment process (Kansas, Blood 88:3259-3287 (1996)), and may prime monocytes for tissue factor and cytokine upregulation Weyrich et al, J. Clin. Invest. 95:2297-2303 (1995)). In the setting of CPB, activation of platelets, as measured by the expression of CD62P, peaks at 2-4 hours after CPB and returns to baseline at 18 hours after CPB (Rinder et al, Anesthesiology 75:563-570 (1991)). Through increased platelet CD62P expression, CPB also causes formation of monocyte-platelet, and to a lesser extent, neutrophil-platelet conjugates, with monocyte-platelet binding increasing over a longer duration and to a greater absolute value than neutrophil-platelet binding (Rinder et al, Blood 79:1201-1205 (1992)).

Cerebral ischemia-reperfusion injury produces a profound inflammatory response characterized by neutrophil, macrophage, and platelet accumulation, up-regulation of adhesion molecules, blood brain barrier destruction, and cytokine production D'Ambrosio et al, Mol. Med. 7:367-382 (2001)). In mice, global cerebral ischemia followed by reperfusion leads to the induction, via P-selectin expression on endothelial cells and platelets, of an inflammatory and prothrombotic state in the cerebral microvasculature (Ishikawa et al, Stroke 34:1777-1782 (2003)). The resulting platelet accumulation is thought to augment the inflammatory response and render the brain more vulnerable to microthrombosis, thus amplifying the ischemic insult.

The gene coding for P-selectin (SELP) spans >50 kb, contains 17 exons and 16 introns, and has been reported to be highly polymorphic (Herrmann et al, Hum. Mol. Genet. 7:1277-1284 (1998)). The SELP 1087G/A SNP results in a non-synonymous amino acid change (serine to asparagine) at codon 290 in the consensus repeat domain. This domain has been shown to be important for the binding of P-selectin to its ligand on leukocytes (Patel et al, J. Cell Biol. 131:1893-1902 (1995), Ruchaud-Sparagano et al, Biochem. J. 332(Pt 2):309-314 (1998)). In a study of 582 subjects with MI and 630 age-matched controls, Tregouet et al (Hum. Mol. Genet. 11:2015-2023 (2002)) reported that the risk for MI associated with the SELP 1087G/A SNP was reduced but differed according to the haplotype background. In the present study of cardiac surgical patients, this SNP was associated with lower levels of platelet activation during CPB. Cognitive dysfunction after surgery may thus be diminished in carriers of this polymorphism by a reduction in the perioperative inflammatory and prothrombotic state.

When interpreting positive findings from any genetic association study, several epidemiological limitations should be taken into account, including inadequate sample size, poorly matched control groups, subgroup analyses, multiple testing, and population stratification (Cardon and Bell, Nat. Rev. Genet. 2:91-99 (2001)). With regard to these concerns, strengths of the present study include a large population of cardiac surgery patients (N=516), despite a 24% loss to follow-up, and a prospective cohort design that reduces the selection bias inherent in case-control designs. Although serum CRP and platelet CD62P data were available only on a subset of patients, it is possible to demonstrate a genotype effect on both CRP level and platelet activation. However, it is possible that the CRP 1059G/C and SELP 1087G/A SNPs are in linkage disequilibrium with other functional (causal) variants not included in this study. This is particularly true for the CRP SNP which is silent at the amino acid level. A much larger study incorporating many more SNPs might be necessary to delineate this effect. Further, the structured association analysis revealed no evidence of population stratification in these data. Although differences in cognitive deficit among racial groups were found a genetic effect that differed between races could not be detected. It is possible that a larger sample of patients with varying ethnicity may have demonstrated such an effect. Finally, all results were adjusted for multiple comparisons using permutation testing.

In conclusion, using a prospective cohort study design, two candidate gene polymorphisms independently associated with a reduction in the incidence of cognitive decline following cardiac surgery were found. Functionally, these CRP and SELP polymorphisms were associated with reductions in serum CRP and platelet activation, respectively, suggesting that therapies aimed at reducing the perioperative inflammatory and prothrombotic state may be beneficial. Moreover, using cardiac surgery as a model of neurological injury, these results provide insight into the biological factors modulating cognitive performance in humans, and further evidence for a genetic basis of cognitive deterioration, which should translate into more precise identification of subjects at risk.

### Example 5

### Experimental Details

The patients enrolled in this study were part of the Perioperative Genetics and Safety Outcomes Study-US (PEGASUS), an ongoing Institutional Review Board approved, prospective, longitudinal study at Duke University Medical Center. Patients undergoing cardiac surgery gave written informed consent to have their clinical and genetic data analyzed in relation to perioperative outcomes.

After excluding patients with ventricular conduction abnormalities (bundle branch blocks), pacemaker, perioperative atrial fibrillation, or receiving perioperative antiarrhythmic drugs in the study period, N=460 patients who underwent cardiac surgery (CABG, valve, or combined CAGB/valve) using cardiopulmonary bypass (CPB) were selected. QTc intervals were measured from 24-hour pre- and postoperative 12-lead ECG by two investigators blinded to genetic data; a prolonged QTc was defined as >440 msec. The number of intraoperative cardioversions upon aortic cross-clamp release was recorded as an index of reperfusion arrhythmias (Walker, Cardiovasc. Res. 22(7):447-455 (1988)).

MALDI-TOF mass spectrometry was used to genotype 45 single-nucleotide polymorphisms (SNPs) in 24 candidate genes modulating pathways implicated in arrhythmia susceptibility (see Table 15). A multivariate regression model, including demographic and procedural covariates, was developed. A two-step strategy (Hoh, Ann. Hum. Genet. 64:413-417 (2000)) was used for genetic analyses—marker selection, followed by clinico-genomic model building. The conservative Bonferroni correction was used to adjust for multiple comparisons.

TABLE 15
LIST OF GENE POLYMORPHISMS
EXAMINED IN THIS STUDY

| PATHWAY | GENE NAME | SNPid |
|---|---|---|
| ion channels | SCN5A (Na channel, Long QT 3) | RS1805124 |
| | KCNQ1 (K channel, voltage-gated IKs) | RS1057128 |
| adrenergic | COMT (catecholOmethyl transferase) | RS165688 |
| tone | VMAT2(SLC18A2-vesicular amine | RS2072362 |
| | transporter2) | |
| | ADRB1 (beta1-AR) | RS1801253 |
| | | RS1801252 |
| | ADRB3 (beta3-AR) | RS4994 |
| | ADRB2 (beta2-AR) | RS1042711 |
| | | RS1042713 |
| | | RS1042714 |
| | | RS1800888 |
| | GNB3 (G-protein beta3 subunit) | RS5443 |
| tissue/matrix | ACE (Angiotensin Converting Enzyme) | SNP030 |
| remodeling | | RS4344 |
| | | RS4291 |
| | AGTR1 (Ag receptor 1) | RS5186 |
| | AGT (Angiotensinogen) | RS4762 |
| | | RS699 |
| | | RS5051 |
| | MMP3 (Matrix metalloproteinase 3) | RS3025058 |
| | MMP9 (Matrix metalloproteinase 9) | RS3918242 |
| inflammation | TNFA (TNF alpha) | RS1800750 |
| | | RS1800629 |
| | | RS361525 |
| | | RS1800610 |
| | TNFRSF1B (TNFR2) (TNF receptor 2) | RS1061622 |
| | IL1B | RS1143634 |
| | | RS1143633 |
| | | RS16944 |
| | IL 1A | RS1800587 |
| | | RS17561 |
| | IL1RN (IL1-Ra) | RS2229235 |
| | | RS315952 |
| | IL10 | RS3021097 |
| | | RS1800872 |
| | IL6 | RS1800795 |
| | | RS1800796 |
| | | RS1800797 |
| | CRP | RS1205 |
| | TGFB1 (TGF beta-1) | RS1800468 |
| | | RS1800469 |
| | | RS1800471 |
| | | RS1982073 |
| | | RS1800472 |
| oxidative | NOS3 (endothelial nitric oxide synthase) | RS2070744 |
| stress | | RS1799983 |
| | | RS1799985 |
| | CAT (catalase) | RS769214 |
| | | RS1001179 |
| | | RS17880664 |

### Results

The QTc interval was significantly prolonged following cardiac surgery with cardiopulmonary bypass (p<0.01), and was associated with a higher incidence of intraoperative reperfusion arrhythmias (p=0.006). Gender, age, procedure and cross-clamp time were independent predictors of QTc prolongation. Moreover, two functionally important SNPs in beta-2 adrenergic receptor (ADRB2) and interleukin-1β (IL1β) genes were independently associated with postoperative QTc prolongation (see Table 16—the rs numbers provided in Tables 15 and 16 are the official designation reference numbers from the NCBI SNP website (see, generally, ncbi.nlm.nih.gov/entrez/query.fcgi and, specifically, ncbi.nlm.nih.gov/SNP/snp_ref.cgi?rs=16944 and ncbi.nlm.nih.gov/SNP/snp_ref.cgi?rs=1800888).

TABLE 16
Combined clinical-genetic multivariable models for postoperative
QTc prolongation in cardiac surgical patients

| | Preop | Postop |
|---|---|---|
| QTc* (msec) | 402 + 29 | 421 + 32 |
| Incidence of Prolonged QTc (%) | 9.30 | 22.3 |
| | F-value | p-value |
| Clinical model (r² = 0.39) | | |
| QTC-preop | 175.51 | <0.0001 |
| Gender | 7.45 | 0.006 |
| Age (y) | 7.76 | 0.006 |
| Procedure | 3.78 | 0.011 |
| Cross-clamp time (min) | 5.27 | 0.023 |
| Race (self-reported) | 0.40 | 0.669 |
| Clinico-genomic model (r² = 0.45) | | |
| 1L1B(rs16944)/ADRB2(rs1800888) | 5.68 | 0.044* |

*Bazett's formula

In conclusion, perioperative QTc is modestly but significantly prolonged following cardiac surgery. This may reflect disrupted electrophysiological stability of the myocardium and thus substrate for triggering malignant arrhythmias. Two functional variants in genes related to inflammation and adrenergic responsiveness are independent risk factors for postoperative QTc prolongation. This finding can be used in perioperative identification and monitoring of high-risk cardiac patients and in the development of novel cardioprotective strategies.

### Example 6

### Experimental Details

### Study Sample

Patients scheduled to undergo elective CABG surgery at Duke University Medical Center during a specific time period were recruited. Exclusion criteria included active liver disease, renal disease (creatinine>2), history of cerebral vascular accident (CVA) or stroke, inability to read, less than seventh grade education, any (DSM)-IV (a psychiatric diagnosis criteria) diagnosis, or treatment for a psychiatric disorder, including depression. (See Table 17.)

TABLE 17
Patient characteristics

| Characeristic | Total Sample | Males | Females | p value |
|---|---|---|---|---|
| Age (yrs) | 61.91 (10.90) | 62.59 (11.04) | 64.35 (10.78) | .02 |
| White race (%) | 87.45 | 90.63 | 80.69 | .0001 |
| Duration of CPB (min) | 117.37 (48.45) | 117.53 (48.49) | 117.00 (48.43) | .88 |
| Cross clamp time (min) | 65.07 (33.76) | 64.61 (32.19) | 66.12 (37.15) | .55 |
| Left Ventricular Ejection Fraction | 54.11 (11.59) | 53.61 (11.53) | 55.23 (11.63) | .05 |
| Bypass Grafts | 2.65 (1.37) | 2.78 (1.33) | 2.35 (1.40) | .0001 |
| History of Hypertension (%) | 61.97 | 57.90 | 71.13 | .0001 |
| History of Diabetes (%) | 28.46 | 24.38 | 37.68 | .0001 |
| ASA class IV (%) | 61.55 | 61.79 | 61.06 | .83 |
| CCS Class IV Angina (%) | 57.67 | 59.50 | 56.81 | .42 |
| Previous myocardial infarction (%) | 45.01 | 48.60 | 36.97 | .001 |
| Preoperative CESD | 12.91 (10.45) | 11.11 (9.59) | 17.23 (11.18) | <.0001 |

Values are means (and standard deviations) or percentages
P values compare the males and females; categorical comparisons are made with exact chi-square test; continuous variables are compared with a Wilcoxon rank sum

### Patient Management

Anesthesia was induced and maintained with midazolam, fentanyl, and isoflurane. All patients underwent nonpulsatile hypothermic (30°-32° C.) CPB. The perfusion apparatus consisted of a Cobe CML membrane oxygenator™ (COBE Chem Labs, Lakewood, Colo.), Sarns 7000 MDX pump™ (3M Inc, Ann Arbor, Mich.), and Pall SP3840 arterial line filter (Pall Biomedical Products Co, Glen Cove, N.Y.). Perfusion was maintained at pump flow rates of 2 to 2.4 L·min⁻¹·m² throughout CPB. The pump was primed with crystalloid, and serial hematocrits were kept at ≧0.18 with packed red blood cell transfusion as necessary. Arterial blood gases were measured every 15 to 30 minutes to maintain arterial carbon dioxide partial pressures of 35 to 40 mm Hg, unadjusted for temperature (a-stat), and oxygen partial pressures of 150 to 250 mm Hg.

### Assessment of Depression

On the day prior to surgery, and at six months and one year after surgery, patients completed a 20-item Center for Epidemiological Studied-Depression questionnaire (CES-D) (Radloff, Applied Psychological Measures 1:385-401 (1977)). The CES-D is a well-researched scale with high internal consistency and test-retest reliability, and scores correlate with clinicians' ratings and with other self-report measures (Blumenthal et al, Lancet 362:604-9 (2003)). Patients rate the degree to which they have experienced a range of symptoms of depression such as “I had crying spells,” “I felt lonely,” and “I was bothered by things that usually don't bother me”. Items are rated on a 4 point Likert scale, and are summed for a total depression score. Scores range from 0 to 60 with higher scores indicating greater depressive symptoms.

The CES-D was used to categorize patients as non-depressed (CES-D<16), mildly depressed (CES-D 16-26) and severely depressed (CESD>=27) at baseline, 6 months, and at 1 year (Blumenthal et al, Lancet 362:604-9 (2003); Radloff, Applied Psychological Measures 1:385-401 (1977); Sabol, Human Genetics 103:273-9 (1998)). For sample size reasons, mildly depressed patients were combined with moderate to severely depressed patients for genotype association testing. Logistic regression was used to assess the relationship between genotype and depression, defined as a binary variable (CESD>=16).

### Genotyping

### DNA Extraction and Analysis

Blood samples were collected from consented patients into 3 mL Vacutainer (Becton Dickinson, Franklin Lakes, N.J.) blood tubes containing 5.4 mg K₂ EDTA and DNA was isolated from lymphocytes using an AutoGen3000 automated platform for isolating genomic DNA of whole blood (AutoGen Inc., Cambridge, Mass.).

### MAO-A

Genotyping to determine allele frequency of a 30 bp variable number tandem repeat (VNTR) mutation in the monoamine oxidase A promoter region (Sabol et al, Hum. Genet. 103:273-79 (1998)) was carried out via polymerase chain reaction (PCR) using primers corresponding to nucleotide positions −1347 to −1367 (5′-ACAGCCTGACCGTGGAGAAG-3′) and −1043 to −1024 (5′-GAACGGACGCTCCATTCGGA-3′) for the 3 repeat allele. Briefly, reaction conditions consisted of 95° C. for 5 minutes, followed by 35 cycles of denaturation at 95° C. for 30 seconds, annealing at 62° C. for 30 seconds, extension at 72° C. for 1 minute, and finally 72° C. for 10 minutes. PCR products were separated by running out on 3% agarose gels or 12% acrylamide gels containing ethidium bromide and visualized with UV light. Repeat variants were confirmed via sequencing of PCR products from representative samples.

### STPR

Genotyping of an insertion/deletion mutation in the serotonin transporter promoter region (STPR) was carried out via polymerase chain reaction (PCR) amplification to determine alleles containing either the short ( 475 bp) or long (518 bp) variant using primers corresponding to nucleotide positions −1406 to −1384 (5′-CTCTGAATGCCAGCACCTAACCC-3′) and −910 to −888 (5′-GAGGGACTGAGCTGGACAACCAC-3′). Briefly, reaction conditions consisted of 95° C. for 5 minutes, followed by 40 cycles of denaturation at 95° C. for 30 seconds, annealing at 62° C. for 30 seconds, extension at 72° C. for 1 minute, and finally 72° C. for 10 minutes. PCR products were separated by running out on 2% agarose gels containing ethidium bromide and visualized with UV light. Since the serotonin transporter promoter region is GC rich, 7-Deaza-2′-dGTP was used in place of dGTP for PCR reactions as previously described (Lesch et al, Science 274:1527-31 (1996)). Short and long variants were confirmed via sequencing of PCR products from representative samples.

### Potential Confounders

Age, gender, and ethnicity are all potential confounders of depression. Age is tested as a covariate in all statistical models. Gender is treated as appropriate for each SNP (discussed individually below).

Several studies have shown that allele frequencies vary as a function of ethnicity in serotonin related genes (Gelernter et al, Am. J. Psych. 155:1332-8 (1998); Williams et al, Neuropsychopharmacology 28:533-41 (2003)). Therefore, self-identified race, which has been shown to account well for genetic cluster membership (Tang et al, Am. J. Hum. Genet. 76:286-275 (2005)), was controlled for in the statistical modeling. Because the sample is heavily Caucasian, race was tested as a binary covariate (white vs. non-white) in the analyses. Interactions between genotype and race were also tested to address whether the effect of genotype variation on depression depends on race.

### Monoamine Oxidase A

MAOA-uVNTR is characterized by the number of tandem repeats, which can range from 2 to 6. There is discrepancy in the literature regarding the appropriate method for combining these categories for analysis purposes (Sabol, Hum. Genet. 103:273-9 (1998); Deckert et al, Hum. Mol. Genet. 8:621-4 (1999); Huang, Neuropsychopharmacology 29:1498-1505 (2004)). Preliminary descriptive data show that, in the initial sample of 1004 patients with MAOA genotyping, 14 different genotypes are represented in the data. Eleven of these genotypes are rare, each occurring in less than 1% of the sample. Ninety four percent of the sample is accounted for with 3 genotypes: the 3 and 4 homogyzotes, and the ¾ heterozygotes. Rather than risk diluting possible effects by inappropriately combining categories, the more conservative approach of analyzing only the 3 and 4 repeats was chosen. While this decision modestly reduces the sample available for analysis, it may also strengthen the observed associations by maintaining homogeneous groups.

Because the MAO gene is located on the X chromosome (Jonsson et al, Psych. Res. 79:1-9 (1998)), only females can be heterozygotes, with the rare exception of Kinefelter males. Therefore, in the analysis men, who are all hemizygous, have two possible genotypes (3 or 4) and women have three possible genotypes (3/3, 3/4, and 4/4). Because of these gender differences, all MAO-A analyses are stratified by gender.

Serotonin transporter promoter

5HTTLPR is characterized by long (L) and short (S) alleles; analyses are based on all three possible genotypes (L/L, L/S, and S/S). Because initial analyses show no gender differences in genotype distribution, males and females were analyzed together. Gender is included as a covariate in the models because of gender differences in depression rates.

### Statistical Modeling

Logistic regression was used to assess the association between genetic variation and post-operative depression, assessed separately at 6-month and 1-year. The analysis was conducted in two parts. First, postoperative depression was considered as the endpoint of interest. Because patients who are depressed at follow-up could be either chronically depressed (defined as CESD>=16 at baseline, and CESD>=16 at followup), or new onset cases (defined as CESD<16 at baseline, but >=16 at followup) a second analysis was performed after eliminating patients who were depressed preoperatively. The MAO-A genotype was tested with a two degree of freedom test in women, using the 4/4 genotype as the reference group, and a one degree of freedom test in men, using the 4 genotype as the reference group. (See Mitchell et al, Am. Heart H. 150(5):1017 (2005), Marcus et al, J. Affect. Disord. 87(2-3):141 (2005), Sloan et al, Psychiatr. Clin. North Am. 26(3):581 (2003).)

### Results

The final sample consisted of 427 patients who had both genotyping and baseline depression testing. The six-month follow-up sample had 420 patients, and the one-year sample had 411. Incidence rates of depression at each time point are shown in Table 18. Rates are highest for both males and females at pre-operative baseline; 6 month and 1 year incidence rates are similar.

TABLE 18
CES-Depression Categories*

| | | No Depression | | Mild Depression | | Mod to Severe Depression | |
|---|---|---|---|---|---|---|---|
| | | (CESD < 16) | | (CESD 16-26) | | (CESD >= 27) | |
| | N | Males | Females | Males | Females | Males | Females |
| Baseline | 437 | 72 | 44 | 21 | 40 | 17 | 7 |
| 6 months | 420 | 84 | 67 | 13 | 20 | 4 | 13 |
| 1 year | 411 | 83 | 68 | 13 | 19 | 4 | 13 |

Values represent the % of patients at each time point in each depression category

At one year after surgery, 17% of males and 32% of females were depressed. 4% of males and 13% of females met the criterion for moderate to severe depression. There were 57 cases of new onset depression at 1 year: 23 women and 34 men.

In women, the MAO-A genotypes are 14% 3/3, 43% 3/4, and 42% 4/4. In men, there are 36% 3's, and 64% 4's. In women, the 5HTTLPR genotypes are 34% LIL, 44% S/L, and 22% S/S. In men, the genotypes are 35% L/L, 46% S/L, and 19% S/S. The 5HTTLPR genotype distributions do not differ statistically between men and women (p=0.53).

Neither candidate SNP is predictive of depression at baseline (MAO-A in males p=0.63; MAO-A in females p=0.43; 5HTTLPR in combined group=0.58)

### MAO-A

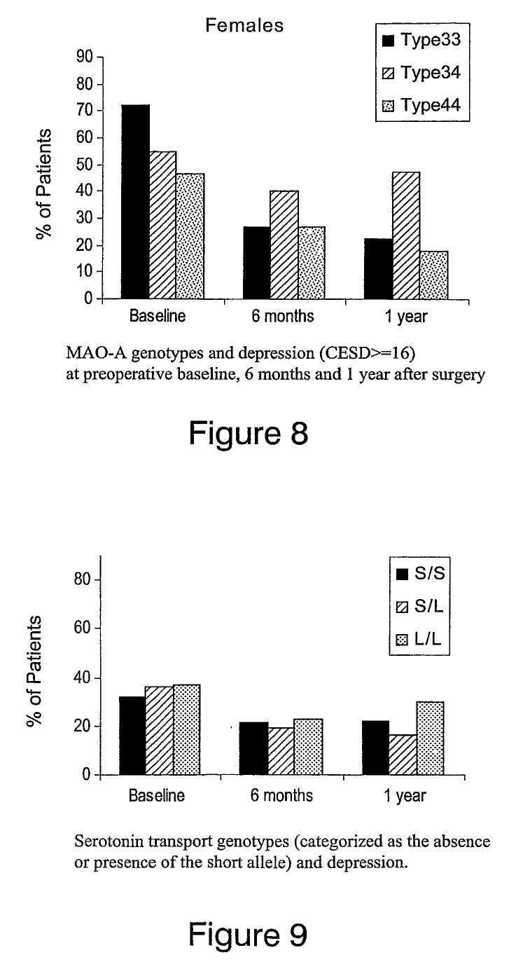

Logistic regression analysis shows that MAO-A genotype is predictive of all depression one year after cardiac surgery in women but not in men, and of new onset depression in women, but not in men. These results are presented in Table 19A. Age was tested in these models and found not significant (p=0.41 and 0.41 for women; p=0.18 and p=0.33 for men). (See also FIG. 8.)

TABLE 19
Association of genotypes and depression-one year results
19A. MAO-A

| | All 1 year depression | | | New Onset depression | | |
|---|---|---|---|---|---|---|
| | Wald chi | Odds ratio | | Wald chi | | |
| | Square | (95% CI) | P value | Square | Odds ratio | P value |
| Females* | | | | | | |
| MAO-34 | 10.17 | 3.67(1.68-8.36) | .0014 | 10.50 | 6.56(2.22-22.45) | .0012 |
| MAO-33 | 0.77 | 1.22(0.24-4.71) | .78 | 1.53 | 3.28(0.40-20.59) | .22 |
| Males** | | | | | | |
| MAO-3 | 0.24 | 0.85(0.44-1.63) | .62 | 0.59 | 1.35(0.63-2.93) | .44 |

19B. MAO-A

| | All six-month depression | | | New Onset depression | | |
|---|---|---|---|---|---|---|
| | Wald chi | Odds ratio | | Wald chi | | |
| | Square | (95% CI) | P value | Square | Odds ratio | P value |
| Females* | | | | | | |
| MAO-34 | 4.11 | 1.95(1.03-3.76) | .04 | 6.04 | 3.03(1.28-7.58) | .01 |
| MAO-33 | 0.12 | 1.21(0.39-3.43) | .73 | 0.55 | 1.76(0.34-7.40) | .46 |
| Males** | | | | | | |
| MAO-3 | 1.56 | 0.64(0.30-1.27) | .21 | 3.28 | 0.60(0.34-1.03) | .07 |

19C. 5HTTLPR

| | All 1 year depression | | | New Onset depression | | |
|---|---|---|---|---|---|---|
| | Wald chi | Odds ratio | | Wald chi | | |
| | Square | (95% CI) | P value | Square | Odds ratio | P value |
| Gender | 10.7578 | 2.24(1.38-3.64) | .001 | 2.042 | 2.13(1.16-3.87) | .01 |
| STPR-Short | 7.342 | 1.95(1.20-3.15) | .007 | 5.99 | 1.55(0.85-2.81) | .15 |

*The reference group for females is 4/4
**The reference group for males is 4

Race is not a significant predictor of 1 year depression in women (p=0.78) and, therefore, is not presented in the final model. Race is a significant predictor of 1 year depression in men, with non-white males having more depression than white males (41% vs. 15%, p=0.003). This finding was followed up in two ways: 1) investigation of interaction terms between genotype and race among men (p=0.60), and 2) limiting the analysis sample a single race. Due to the small sample size of non-whites, the analysis was repeated in whites only (genotype p=0.59). These follow-up analyses suggest that there is no association between depression and MAOA genotype in men, either adjusted or unadjusted for race. Age is not a significant predictor in these models.

The six-month analyses have similar results, but not as strong. The association of the ¾ genotype in women with all 6 month depression (p=0.04), and with new onset depression (p=0.01) are both significant. There is a borderline significant effect for men (p=0.07), suggesting a potentially protective effect of the 3 genotype compared to the 4's. (See Table 19B above)

### Serotonin Transporter Promoter

Initial analyses were done combining both sexes, and using all 3 genotypes. Logistic regression shows a significant sex effect at one year (p=0.004) and a significant overall genotype effect, (p=0.04), but no race effect (p=0.09). To follow-up on the genotype effect, genotypes were characterized as either having either 1 or two copies of the short allele (S/S and S/L), or no copies (L/L) in a model which also adjusts for sex. This analysis was conducted for all 1 year depression, and for new onset depression. The p value for genotype for all 1-year depression is 0.007, indicating that the L/L combination is the risk genotype. There is no significant association of genotype with new onset depression (p=0.15). No significant results were found at 6 months. One year results are shown in Table 19C above. (See also FIG. 9.)

In conclusion, the findings described above indicate that MAO-A is associated with both chronic and new onset depression in females, but not males, at both six months and one year after CABG surgery. Because the 3/4 heterozygote is the genotype which apparently confers risk, only patients with two MAO-A alleles are able to manifest this genotype—thus limiting the effect to females. This finding is different from those reports which speculate that alleles with repeats that are more efficiently transcribed are important in explaining personality and behavioral traits for conferring risk or protection. In contrast, and as indicated above, 4's are not a risk (“efficient” transcribers), and 3's are not a risk (not efficient), but the combination of 3 and 4 is a risk. This observation is consistent with the persistent finding that females exhibit greater levels of depression than males.

In general, women experience depression about twice as often as men. There are a number of potential explanations in the literature about why females show different depression patterns than males, many of which focus on reproductive events (puberty, pregnancy, miscarriage, menopause), and all of which have both hormonal and psychosocial components. Many women also face additional stresses such as responsibilities both at work and home, single parenthood, and caring for children and for aging parents (NIMH)

Previous research shows women are more likely than men to be depressed both before and after CABG surgery (Phillips Bute et al, Psych. Med. 65:944-51 (2003)), and are also at risk for poorer outcomes (Connerney et al, Lancet 358:1766-71 (2001)) and impaired functional and psychosocial quality of life (Phillips Bute et al, Psych. Med. 65:944-51 (2003)). In addition to hormonal and psychosocial factors, there is a reasonable expectation that there is a genetic component as well, as in other complex multi-causal conditions.

Despite previous reports that serotonin is associated with mood disorders, including clinical depression, neither candidate SNP in the current study is predictive of baseline depression in the sample. This finding can be perhaps be explained by the anticipation of impending surgery, which could temporarily elevate CES-D scores, and, therefore reflect, transitory cases of depression which resolve when the surgery is complete. Genetic variability, as captured by these two SNPs, does not address this transitory depression that remits after surgery. In contrast, patients who are depressed at 1 year are either chronically depressed or have new onset depression, some of which can be explained by genetic variability.

There is considerable evidence for the monoamine theories of depression, which have been extensively reviewed. Thousands of studies have been conducted to investigate the role of neurotransmitter systems as the biological basis for depression, and all of the antidepressant drugs currently in use modulate monoamine neurotransmission (Wong et al, Nat. Rev. Drug Disc. 3:136-51 (2004)).

It should be noted that not all theories of depression are biochemical in nature. For example, patients with depression have reduced grey matter in the prefrontal cortex and hippocampus, and resultant reduction in neuronal complexity and connectivity (Castren, Nat. Rev. Neur. 6:241-6 (2005)). Depression might actually be preceded by functional deficits, such as memory impairment. Anti-depressants, which have been shown to increase the production of new neurons in the rodent hippocampus, might work through morphological and physiological reorganization of neuronal connections, leading to improvements in neuronal information processing and recovery of mood. Wong (Wong et al, Nat. Rev. Drug Disc. 3:136-51 (2004)) predicts a paradigm shift away from the biochemistry of monoamines toward identification of pharmacogenetic targets that mediate antidepressant effects on neuronal functions.

There is some suggestion that genetic variability and environmental factors interact in complex ways, addressing the question of why stressful experiences lead to depression in some people but not in others (Caspi et al, Science 301:386-9 (2003)). Evidence has been provided that the serotonin transport gene, in interaction with environmental factors, affects the manner in which an individual responds to environmental insults, thus moderating the effect of stressful events on depression (Caspi et al, Science 301:386-9 (2003)). Alternatively, interactions between stressful life events and genetic risk factors may increase the risk for depression or other psychiatric illness by causing individuals to select themselves into high risk environments (Kendler and Karkowski-Shuman, Psych. Med. 27:539-47 (1997)). It has been found that lower expression of the MAOA-uVNTR polymorphism is associated with a history of abuse in male subjects, who manifested high levels impulsivity (Huang, Neuropsychopharmacology 29:1498-1505 (2004)), suggesting that these lower expression levels sensitized the boys to the effects of early abuse experiences.

Blumenthal et al (Lancet 362:604-9 (2003)) found that patients who are depressed six months after CABG surgery are at risk for early mortality. The studies described herein result in the identification of genotypes that can help identify these patients. In addition, a genotype that can help predict new onset depression has been identified. Although in most circumstances the best predictor of future depression is past depression, in a sample such as that described here, in which some patients are expected to remit, preoperative depression may not be the most useful predictive tool.

There are multiple behavioral and physiological factors that can help explain the relationship between depression and CAD, including smoking, drinking, treatment adherence, exercise, heart rate variability (Carney et al, Psych. Med. 66:466-74 (2004)), inflammation, and medical comorbidities. (Lett et al, Psych. Med. 66:305-0.15 (2004)). The genetic possibility is one more in a complex set of relationships.

While it is not known if treatment of depression improves outcomes (Bush et al, Post-Myocardial Infarction Depression. Summary, Evidence Report/Technology Assessment: Number 123. AHRQ Publication Number 05-EO18-1 Agency for Healthcare Research and Quality, Rockville, Md. (2005)), some well-researched treatments for depression in addition to medication include diet and exercise, all of which seem helpful in relieving depression at least to some degree.

There are several possible limitations of the present study. First, it is not known who received treatment for depression after surgery, and whether or not intervening treatment affected the incidence rate. There is a possibility that some patients who would otherwise be classified as depressed at follow-up received treatment and thus were not seen as depressed. If this did in fact happen, it may have reduced the ability to detect an association. Second, the implications of excluding patients who were clinically depressed at baseline may have affected the analysis of chronic depression. It would not, however, affect the findings regarding new onset depression as they would have been excluded from analysis.

Despite advances and improved outcomes following CABG surgery, depression remains a particularly devastating concern, especially for women. Knowledge of genetic risk factors for depression may help patients and their physicians make informed treatment decisions, and alert them to be watchful for symptoms of depression within the first year after surgery. The prognostic importance of clinical depression after surgery has been demonstrated, and improved ability to anticipate new onset depression could prove valuable as a tool for reducing adverse outcomes associated with the CABG procedure.

### Example 7

### Experimental Details

The Perioperative Genetics and Safety Outcomes Study-US (PEGASUS) is an ongoing prospective and longitudinal study, including over 3300 consenting patients scheduled for cardiac surgery.

Data was prospectively collected as part of a quality assurance database, including demographic, historical, clinical, laboratory, and investigational test information, resource utilization, and adverse outcome. All outcomes are prespecified and defined by protocol.

736 patients undergoing aortocoronary surgery with cardiopulmonary bypass were studied. Clinical co-variates previously associated with bleeding were recorded and DNA was isolated from preoperative blood. MALDI-TOF mass spectroscopy was used for genotype analysis. Multivariable linear regression modeling related clinical and genetic predictors to bleeding from the thorax and mediastinum.

### Results

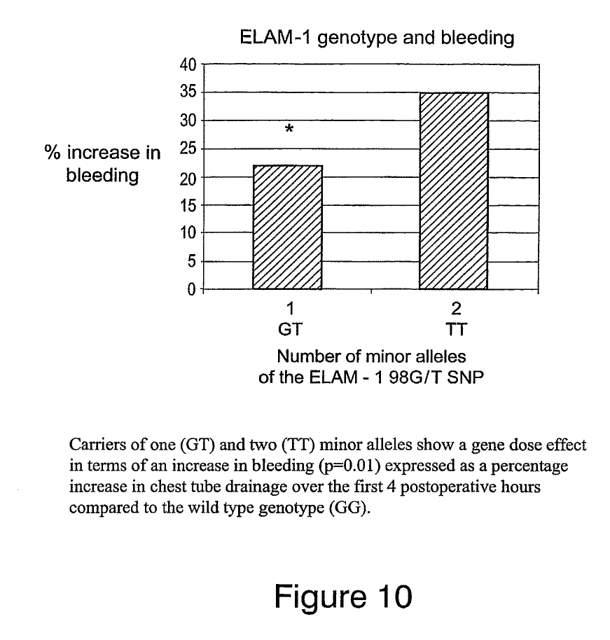

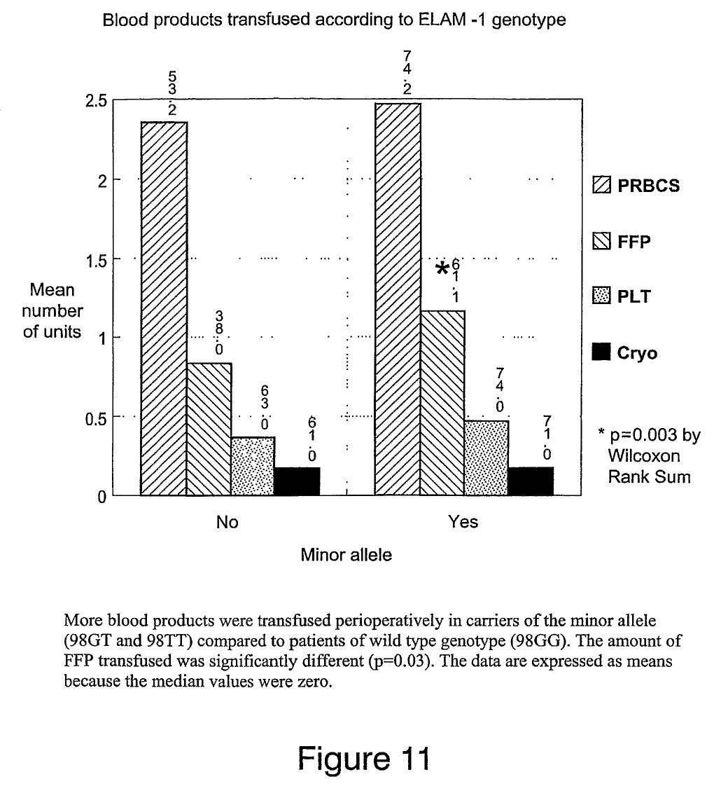

The 98G/T SNP of the E-selectin (ELAM-1) gene was independently associated with bleeding after cardiac surgery (p=0.002), after adjusting for clinical predictors (patient size and preoperative hemoglobin concentration). There was a gene dose effect according to the number of minor alleles in the genotype, as shown in FIG. 10. Carriers of the minor allele, heterozygotes (GT) and homozygotes (TT) bled 20% more and 35% more than homozygous wild type respectively (P=0.01). (See FIG. 11.)

The results of the genetic and clinical multivariate models are found in Table 20.

TABLE 20

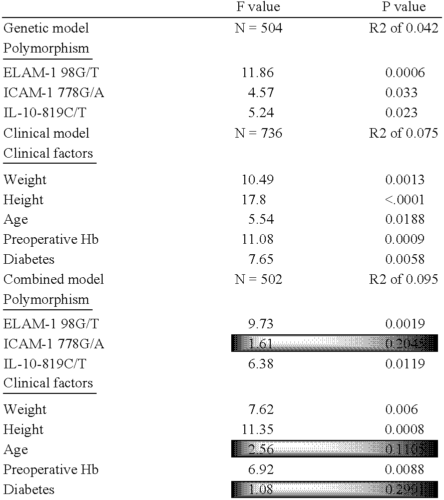

Table 20 legend
Results of multivariate linear regression models testing the association of inflammatory and adhesion molecule polymorphisms and clinical covariates with 4 hour postoperative chest tube drainage. Variables identified through independent assessments of clinical (A) and genetic (B) models were entered into a combined clinical and genetic variables model (C).

In conclusion, the above-described study resulted in the identification of an E-selectin/ELAM-1 polymorphism that was associated with bleeding after cardiac surgery, that is independent of and additive to clinical predictors of bleeding. Preoperative testing for carriers of the 98T allele of this polymorphism would result in the identification patients with increased risk of bleeding and transfusion after cardiac surgery.

All documents and other information sources cited above are hereby incorporated in their entirety by reference.

What is claimed is:

1. A method of identifying a patient at risk of perioperative, periprocedure or emergency-related myocardial injury comprising assaying DNA from said patient for a polymorphism in at least one gene selected from the group consisting of the C-Reactive Protein (CRP) gene, Lipopolysaccharide Binding Protein (LBP) gene, Interleukin 6 (IL6) gene and Intercellular Adhesion Molecule 1 (ICAM1) gene,
wherein the presence of a CRP 1846C/T polymorphism, a LBP 19983T/C polymorphism, an IL6-572G/C polymorphism or an ICAM1 1462A/G polymorphism, or the presence of another polymorphism within the same haplotype blocks, or in linkage disequilibrium therewith, is associated with said myocardial injury risk.

2. A method of identifying a patient at reduced risk of perioperative, periprocedure or emergency-related myocardial injury comprising assaying DNA from said patient for a polymorphism in at least one gene selected from the group consisting of the Endothelial Leukocyte Adhesion Molecule 1 (ELAM1) gene and Catalase (CAT) gene,
wherein the presence of a ELAM1 98G/T polymorphism or a CAT-844 G/T polymorphism, or the presence of another polymorphism within the same haplotype blocks, or in linkage disequilibrium therewith, is associated with said reduced myocardial injury risk.

3. A method of identifying a female patient at risk of a perioperative, periprocedure or emergency-related major adverse cardiac event (MACE) comprising assaying DNA from said patient for a polymorphism in the Selectin P (SELP) gene,
wherein the presence of a SELP 1902A/G polymorphism, or the presence of another polymorphism within the same haplotype block, or in linkage disequilibrium therewith, is associated with said MACE risk.

4. A method of identifying a patient at risk of perioperative, periprocedure or emergency-related cognitive decline comprising assaying DNA from said patient for a polymorphism in at least one gene selected from the group consisting of the CRP gene and SELP gene,
wherein the presence of a CPR 1059G/C polymorphism or a SELP 1087G/A polymorphism, or the presence of another polymorphism within the same haplotype blocks, or in linkage disequilibrium therewith, is associated with said cognitive decline risk.

5. A method of identifying a patient at risk of perioperative, periprocedure or emergency-related arrhythmia comprising assaying DNA from said patient for a polymorphism in at least one gene selected from the group consisting of the P-2 adrenergic receptor (ADRB2) gene and interleukin 1β (IL1β) gene,
wherein the presence of an ADRB2 (rs1800888) polymorphism or an IL1β (rs16944) polymorphism, or the presence of another polymorphism within the same haplotype blocks, or in linkage disequilibrium therewith, is associated with said arrhythmia risk.

6. A method of identifying a patient at risk of perioperative, periprocedure or emergency-related depression comprising assaying DNA from said patient for a polymorphism in the upstream regulatory region of the monoamine oxidase A (MAOA) gene or the 5′ flanking regulatory region of the serotonin transporter gene,
wherein the presence of a 30 bp variable number tandem repeat (VNTR) mutation in the MAOA promoter region, MAOA-uVNTR, or the presence of another polymorphism within the same haplotype block, or in linkage disequilibrium therewith, is associated with chronic and new onset depression risk in female patients and a 44-base pair insertion/deletion polymorphism in the 5′ flanking regulatory region of the serotonin transporter gene, 5HTTLRP, or the presence of another polymorphism within the same haplotype block, or in linkage disequilibrium therewith, is associated with chronic depression risk in female and male patients.

7. A method of identifying a patient at risk of perioperative, periprocedure or emergency-related bleeding comprising assaying DNA from said patient for a polymorphism in the ELAM1 gene,
wherein the presence of an ELAM1 98G/T polymorphism, or the presence of another polymorphism within the same haplotype block, or in linkage disequilibrium therewith, is associated with said bleeding risk.

8. A kit comprising a probe or primer suitable for use in detecting a polymorphism of claim 1.

9. A kit comprising a probe or primer suitable for use in detecting a polymorphism of claim 2.

10. A kit comprising a probe or primer suitable for use in detecting the polymorphism of claim 3.

11. A kit comprising a probe or primer suitable for use in detecting a polymorphism of claim 4.

12. A kit comprising a probe or primer suitable for use in detecting a polymorphism of claim 5.

13. A kit comprising a probe or primer suitable for use in detecting a polymorphism of claim 6.

14. A kit comprising a probe or primer suitable for use in detecting the polymorphism of claim 7.

15. A probe or primer suitable for use in detecting a polymorphism of claim 1 bound to a solid support.

16. A probe or primer suitable for use in detecting a polymorphism of claim 2 bound to a solid support.

17. A probe or primer suitable for use in detecting the polymorphism of claim 3 bound to a solid support.

18. A probe or primer suitable for use in detecting a polymorphism of claim 4 bound to a solid support.

19. A probe or primer suitable for use in detecting a polymorphism of claim 5 bound to a solid support.

20. A probe or primer suitable for use in detecting a polymorphism of claim 6 bound to a solid support.

21. A probe or primer suitable for use in detecting the polymorphism of claim 7 bound to a solid support.


SEQUENCE LISTING (50 sequences)

SEQ ID NO: 1 - DNA, 30 bp, Artificial Sequence
  Description of Artificial Sequence Synthetic primer
acgttggatgagtgcatctgaatgggcaag

SEQ ID NO: 2 - DNA, 30 bp, Artificial Sequence
  Description of Artificial Sequence Synthetic primer
acgttggatgtgctgaccaagaataaggcc

SEQ ID NO: 3 - DNA, 22 bp, Artificial Sequence
  Description of Artificial Sequence Synthetic primer
catctgaatgggcaagaaggag

SEQ ID NO: 4 - DNA, 30 bp, Artificial Sequence
  Description of Artificial Sequence Synthetic primer
acgttggatgattttctcctcagaggctcc

SEQ ID NO: 5 - DNA, 30 bp, Artificial Sequence
  Description of Artificial Sequence Synthetic primer
acgttggatgtgtctgtattgagggtgtgg

SEQ ID NO: 6 - DNA, 20 bp, Artificial Sequence
  Description of Artificial Sequence Synthetic primer
tgcaattgacagagagctcc

SEQ ID NO: 7 - DNA, 30 bp, Artificial Sequence
  Description of Artificial Sequence Synthetic primer
acgttggatgctgcttcccaaaacggaaag

SEQ ID NO: 8 - DNA, 30 bp, Artificial Sequence
  Description of Artificial Sequence Synthetic primer
acgttggatggctgagagaaactgtgaagc

SEQ ID NO: 9 - DNA, 24 bp, Artificial Sequence
  Description of Artificial Sequence Synthetic primer
tatttcaagcctaaacctttgggt

SEQ ID NO: 10 - DNA, 84 bp, Artificial Sequence
  Description of Artificial Sequence Synthetic primer
acgttggatggccatcttgtttgccacatgacgttggatggtttgtcaatcccttggctcttgtttgccacatggagagagact

SEQ ID NO: 11 - DNA, 84 bp, Artificial Sequence
  Description of Artificial Sequence Synthetic primer
acgttggatggaaatgtgaacatgtgggacacgttggatgaggacattaggactgaagggtgtgaacatgtgggactttgtgct

SEQ ID NO: 12 - DNA, 80 bp, Artificial Sequence
  Description of Artificial Sequence Synthetic primer
acgttggatgtttcacattgctcaggaagcacgttggatgatctgcacttgtgatcatgggctcaggaagctaaaaggtg

SEQ ID NO: 13 - DNA, 83 bp, Artificial Sequence
  Description of Artificial Sequence Synthetic primer
acgttggatgttgggagaaaggaaggcatgacgttggatgttctaccacctgaactaggctttttacatatgagccttcaatg

SEQ ID NO: 14 - DNA, 81 bp, Artificial Sequence
  Description of Artificial Sequence Synthetic primer
acgttggatggaacagaaagcaggacaagcacgttggatgaggcggcagactcaaaactgcgccttcatccgctcagacag

SEQ ID NO: 15 - DNA, 83 bp, Artificial Sequence
  Description of Artificial Sequence Synthetic primer
acgttggatgtgggatgttaaccagaagacacgttggatgaattgacatttggtccttgcctgaggaacaaccaactagttgc

SEQ ID NO: 16 - DNA, 82 bp, Artificial Sequence
  Description of Artificial Sequence Synthetic primer
acgttggatgagcctcaatgacgacctaagacgttggatggattgtgcaatgtgacgtcctttccccctagttgtgtcttgc

SEQ ID NO: 17 - DNA, 79 bp, Artificial Sequence
  Description of Artificial Sequence Synthetic primer
acgttggatgacgccttgaagtaactgcacacgttggatgtcttctgtgttctggctctccaggcagtctacaacagcc

SEQ ID NO: 18 - DNA, 83 bp, Artificial Sequence
  Description of Artificial Sequence Synthetic primer
acgttggatgacgccttgaagtaactgcacacgttggatgtcttctgtgttctggctctcaagtaactgcacgaaatttgagg

SEQ ID NO: 19 - DNA, 83 bp, Artificial Sequence
  Description of Artificial Sequence Synthetic primer
acgttggatggaaagatgtgcgctgataggacgttggatgcttgccacatctctttctgcgggagggatggagagaaaaaaac

SEQ ID NO: 20 - DNA, 83 bp, Artificial Sequence
  Description of Artificial Sequence Synthetic primer
acgttggatggatttgtgtgtaggaccctgacgttggatgggtccccaaaagaaatggagaccctggaggctgaaccccgtcc

SEQ ID NO: 21 - DNA, 80 bp, Artificial Sequence
  Description of Artificial Sequence Synthetic primer
acgttggatgctcccagttctagttctatcacgttggatgttgcctccatttcttttgggttcctgcatcctgtctggaa

SEQ ID NO: 22 - DNA, 79 bp, Artificial Sequence
  Description of Artificial Sequence Synthetic primer
acgttggatgacacaaatcagtcagtggccacgttggatgatcaaggatacccctcacacgaagacccccctcggaatc

SEQ ID NO: 23 - DNA, 79 bp, Artificial Sequence
  Description of Artificial Sequence Synthetic primer
acgttggatggtcctcatatcaatgtggccacgttggatgctatggttctccattcctttgggacaagacatggttttt

SEQ ID NO: 24 - DNA, 77 bp, Artificial Sequence
  Description of Artificial Sequence Synthetic primer
acgttggatgaaaaatttagccaggcgtggacgttggatgggttcaagcaattctcctgcccaggcgtggtggcgca

SEQ ID NO: 25 - DNA, 82 bp, Artificial Sequence
  Description of Artificial Sequence Synthetic primer
acgttggatggcctccatgtctttctgttcacgttggatgtcttcatccccatctgagacgttcttactttcactcaccagc

SEQ ID NO: 26 - DNA, 83 bp, Artificial Sequence
  Description of Artificial Sequence Synthetic primer
acgttggatgttcaatgttggctccacctgacgttggatggcattccacattattgggccgctccacctgtcatttctcttgt

SEQ ID NO: 27 - DNA, 85 bp, Artificial Sequence
  Description of Artificial Sequence Synthetic primer
acgttggatggcaaaagcagtgagcggatgacgttggatgtctccagctgtgcagtgtcagtgaacacagtccatggttccttca

SEQ ID NO: 28 - DNA, 80 bp, Artificial Sequence
  Description of Artificial Sequence Synthetic primer
acgttggatgtacagtacatggttccctgcacgttggatgacaggcatagcatcacttccggtgagggctggacattgca

SEQ ID NO: 29 - DNA, 78 bp, Artificial Sequence
  Description of Artificial Sequence Synthetic primer
acgttggatgatgccaagagaatggccacacgttggatgcctgcttggcaggttggcaaagagaatggccactggtca

SEQ ID NO: 30 - DNA, 78 bp, Artificial Sequence
  Description of Artificial Sequence Synthetic primer
acgttggatgatccactcaaggctcccttgacgttggatgttggcagcaggagcagcaagggctcccttgcccacagg

SEQ ID NO: 31 - DNA, 78 bp, Artificial Sequence
  Description of Artificial Sequence Synthetic primer
acgttggatgtgttgttagccagactgagcacgttggatgaaggcaatgagctgaagacctccagcttgggtgtgggc

SEQ ID NO: 32 - DNA, 80 bp, Artificial Sequence
  Description of Artificial Sequence Synthetic primer
acgttggatgatttcccaggaacctctgtgacgttggatgatacgctgtgcaccagaatgtaccaacagaaccaccttcc

SEQ ID NO: 33 - DNA, 84 bp, Artificial Sequence
  Description of Artificial Sequence Synthetic primer
acgttggatgtggcctattagcaccaaaacacgttggatgagacatcccaatatggtgggttaccttgcatattgaattgctcc

SEQ ID NO: 34 - DNA, 80 bp, Artificial Sequence
  Description of Artificial Sequence Synthetic primer
acgttggatggatccggtgtttgcggaatcacgttggatgagggaaaagtttctgggcagcggaatcaggaggggcgggc

SEQ ID NO: 35 - DNA, 78 bp, Artificial Sequence
  Description of Artificial Sequence Synthetic primer
acgttggatgccttcagcagattctccttcacgttggatgttgctggacttctctttgggtcacagcgaggtgagccc

SEQ ID NO: 36 - DNA, 79 bp, Artificial Sequence
  Description of Artificial Sequence Synthetic primer
acgttggatgctccgatgatacacggctgaacgttggatgaggttgttgacacaagagaggatacacggctgactcccc

SEQ ID NO: 37 - DNA, 83 bp, Artificial Sequence
  Description of Artificial Sequence Synthetic primer
acgttggatgaaggaaacaggagaccaacgacgttggatggaggataaaggacaagctgcgaccaacgtgtaagtttcacttc

SEQ ID NO: 38 - DNA, 79 bp, Artificial Sequence
  Description of Artificial Sequence Synthetic primer
acgttggatgacattccccttccacgcttgacgttggatgtagaggtcttttgaccacccgaatggaggagggtgtctg

SEQ ID NO: 39 - DNA, 77 bp, Artificial Sequence
  Description of Artificial Sequence Synthetic primer
acgttggatgtgtccaaggagctgcaggcacgttggatgtcggtgctctggccgagcatgcggacatggaggacgtg

SEQ ID NO: 40 - DNA, 79 bp, Artificial Sequence; identical to SEQ ID NO: 38
  Description of Artificial Sequence Synthetic primer

SEQ ID NO: 41 - DNA, 84 bp, Artificial Sequence
  Description of Artificial Sequence Synthetic primer
acgttggatggcagaggaagctggagaaagacgttggatgtcgggtgttccggcaaactgctggagaaagggcctcctctcttt

SEQ ID NO: 42 - DNA, 82 bp, Artificial Sequence
  Description of Artificial Sequence Synthetic primer
acgttggatgacaatgttgtgatgggtgccacgttggatgatctatgtcgggcaagtcaccaagattattaacttcttcccc

SEQ ID NO: 43 - DNA, 49 bp, Artificial Sequence
  Description of Artificial Sequence Synthetic primer
ctggagaccactcccatcctttctgatgtggccatcacattcgtcagat

SEQ ID NO: 44 - DNA, 78 bp, Artificial Sequence
  Description of Artificial Sequence Synthetic primer
acgttggatgaaacggtcgcttcgacgtgcacgttggatgatccctttggtgctcacgtggctgcaggccccagatga

SEQ ID NO: 45 - DNA, 80 bp, Artificial Sequence
  Description of Artificial Sequence Synthetic primer
acgttggatgagcggctgcatgacattgagacgttggatggtccctagattgtgtgactctgagagcaaaggtgaggctg

SEQ ID NO: 46 - DNA, 79 bp, Artificial Sequence
  Description of Artificial Sequence Synthetic primer
acgttggatgagtttccctagtcccccatgacgttggatgagtcagcagagagactagggcatcaagctcttccctggc

SEQ ID NO: 47 - DNA, 20 bp, Homo sapiens
acagcctgaccgtggagaag

SEQ ID NO: 48 - DNA, 20 bp, Homo sapiens
gaacggacgctccattcgga

SEQ ID NO: 49 - DNA, 23 bp, Homo sapiens
ctctgaatgccagcacctaaccc

SEQ ID NO: 50 - DNA, 23 bp, Homo sapiens
gagggactgagctggacaaccac